SEQUENCE LISTING (29 sequences)

SEQ ID NO: 1 - protein, 728 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLHCTNVTISSTNGSTANVTMREEMKNCSFNTTTVIRDKIQKEYALFYKLDIVPIEGKNTNTGYRLINCNTSVITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPGNNTRRSINIGPGRAFYATGAIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPGGGDMRDNWRSELYKYKVVEIKPLGVAPTDAKRRVVERNKRAVGIGAVFLGFLGAAGSTMGAASITLTVQARQLLSGIVQQQSNLLRAIEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALDSWNNLWNWFNITNWLWYIKIFIMIVGGLIGLRIIFIVLSIVNRVRQGYSPLSFQTLIPNPRGPDRPGGIEEEGGEQDRDRS

SEQ ID NO: 2 - protein, 728 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLHCTNVTISSTNGSTANVTMREEMKNCSFNTTTVIRDKIQKEYALFYKLDIVPIEGKNTNTGYRLINCNTSVTLQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPGNNTRRSINIGPGRAFYATGAIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPGGGDMRDNWRSELYKYKVVEIKPLGVAPTDAKRRVVERNKRAVGIGAVFLGFLGAAGSTMGAASITLTVQARQLLSGIVQQQSNLLRAIEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALDSWNNLWNWFNITNWLWYIKIFIMIVGGLIGLRIIFIVLSIVNRVRQGYSPLSFQTLIPNPRGPDRPGGIEEEGGEQDRDRS

SEQ ID NO: 3 - protein, 721 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLHCTNVTSVNTTGDREGLKNCSFNMTTELRDKRQKVYSLFYRLDIVPINENQGSEYRLINCNTSAITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQAFYATGDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPGGGDMRDNWRSELYKYKVVEIKPLGVAPTDAKRRVVERNKRAVGIGAVFLGFLGAAGSTMGAASITLTVQARQLLSGIVQQQSNLLRAIEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALDSWNNLWNWFNITNWLWYIKIFIMIVGGLIGLRIIFIVLSIVNRVRQGYSPLSFQTLIPNPRGPDRPGGIEEEGGEQDRDRS

SEQ ID NO: 4 - protein, 721 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLHCTNVTSVNTTGDREGLKNCSFNMTTELRDKRQKVYSLFYRLDIVPINENQGSEYRLINCNTSAITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQAFYATGDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIPGVITCKSDITGLLLLRDGGDTTDNTEIFRPGGGDMRDNWRSELYKYKVVEIKPLGVAPTDAKRRVVERNKRAVGIGAVFLGFLGAAGSTMGAASITLTVQARQLLSGIVQQQSNLLRAIEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALDSWNNLWNWFNITNWLWYIKIFIMIVGGLIGLRIIFIVLSIVNRVRQGYSPLSFQTLIPNPRGPDRPGGIEEEGGEQDRDRS

SEQ ID NO: 5 - protein, 720 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLDCSTYNNTHNISKEMKICSFNMTTELRDKKRKVNVLFYKLDLVPLTNSSNTTNYRLISCNTSTITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQSFYATGEIVGNIREAHCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPGGGDMRDNWRSELYKYKVVEIKPLGVAPTDAKRRVVERNKRAVGIGAVFLGFLGAAGSTMGAASITLTVQARQLLSGIVQQQSNLLRAIEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALDSWNNLWNWFNITNWLWYIKIFIMIVGGLIGLRIIFIVLSIVNRVRQGYSPLSFQTLIPNPRGPDRPGGIEEEGGEQDRDRS

SEQ ID NO: 6 - protein, 720 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLDCSTYNNTHNISKEMKICSFNMTTELRDKKRKVNVLFYKLDLVPLTNSSNTTNYRLISCNTSTITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQSFYATGEIVGNIREAHCNISGRNWSEAVNQVKKKLKEHFPHKNISFQSSSGGDLEITTHSFNCGGEFFYCNTSGLFNDTISNATIMLPCRIKQIINMWQEVGKAMYAPPIAGNITCKSDITGLLLLRDGGDTTDNTEIFRPGGGDMRDNWRSELYKYKVVEIKPLGVAPTDAKRRVVERNKRAVGIGAVFLGFLGAAGSTMGAASITLTVQARQLLSGIVQQQSNLLRAIEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALDSWNNLWNWFNITNWLWYIKIFIMIVGGLIGLRIIFIVLSIVNRVRQGYSPLSFQTLIPNPRGPDRPGGIEEEGGEQDRDRS

SEQ ID NO: 7 - protein, 722 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLQCTNYAPNLLSNMRGELKQCSFNMTTELRDKKRKVNVLFYKLDLVPLTNSSNTTNYRLISCNTSTITQACPKVTFDPIPIHYCAPAGYAILKCNNKTFNGKGPCNNVSTVQCTHGIKPVVSTQLLLNGSLAEEEIVIRSKDLSDNAKIIIVQLNTPVQINCTRPNNNTRKSIRIGPGQSFYATGEIIGDIRMAHCNVSKATWNEAVNQVKKKLKEHFPHKNISFQSSSGGDLEITTHSFNCGGEFFYCNTSGLFNDTISNATIVLPCRIKQIINMWQEVGKAMYAPPIQGVIRCVSNITGLLLLRDGGDTTDNTEIFRPGGGDMRDNWRSELYKYKVVEIKPLGVAPTDAKRRVVERNKRAVGIGAVFLGFLGAAGSTMGAASITLTVQARQLLSGIVQQQSNLLRAIEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALDSWNNLWNWFNITNWLWYIKIFIMIVGGLIGLRIIFIVLSIVNRVRQGYSPLSFQTLIPNPRGPDRPGGIEEEGGEQDRDRS

SEQ ID NO: 8 - protein, 722 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLHCTNVTPNLNTTGDREGLKNCSFNMTTELRDKRQKVYSLFYRLDIVPINENQGSEYRLINCNTSAITQACPKVTFDPIPIHYCAPAGYAILKCNNKTFNGKGPCNNVSTVQCTHGIKPVVSTQLLLNGSLAEEEIVIRSKDLSDNAKIIIVQLNKSVQINCTRPNNNTVKSIRIGPGQAFYYFGDIIGDIRMAYCNISGRNWSEAVNQVKKKLKEHFPHKNISFQSSSGGDLEITTHSFNCGGEFFYCNTSGLFNDTISNDSIMLPCRIKQIINMWQEVGKAIYAPPIPGVITCKSDITGLLLLRDGGDTTDNTEIFRPGGGDMRDNWRSELYKYKVVEIKPLGVAPTDAKRRVVERNKRAVGIGAVFLGFLGAAGSTMGAASITLTVQARQLLSGIVQQQSNLLRAIEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALDSWNNLWNWFNITNWLWYIKIFIMIVGGLIGLRIIFIVLSIVNRVRQGYSPLSFQTLIPNPRGPDRPGGIEEEGGEQDRDRS

SEQ ID NO: 9 - protein, 649 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLHCTNVTISSTNGSTANVTMREEMKNCSFNTTTVIRDKIQKEYALFYKLDIVPIEGKNTNTGYRLINCNTSVITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPGNNTRRSINIGPGRAFYATGAIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPGGGDMRDNWRSELYKYKVVEIKPLGVAPTDCKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICCTAVPWNISWSNKSKEEIWENMTWMQWDREINNYTNTIYRLLEESQNQQENNEKDLLALD

SEQ ID NO: 10 - protein, 649 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLHCTNVTISSTNGSTANVTMREEMKNCSFNTTTVIRDKIQKEYALFYKLDIVPIEGKNTNTGYRLINCNTSVTLQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPGNNTRRSINIGPGRAFYATGAIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPGGGDMRDNWRSELYKYKVVEIKPLGVAPTDCKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICCTAVPWNISWSNKSKEEIWENMTWMQWDREINNYTNTIYRLLEESQNQQENNEKDLLALD

SEQ ID NO: 11 - protein, 642 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLHCTNVTSVNTTGDREGLKNCSFNMTTELRDKRQKVYSLFYRLDIVPINENQGSEYRLINCNTSAITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQAFYATGDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPGGGDMRDNWRSELYKYKVVEIKPLGVAPTDCKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICCTAVPWNISWSNKSKEEIWENMTWMQWDREINNYTNTIYRLLEESQNQQENNEKDLLALD

SEQ ID NO: 12 - protein, 642 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLHCTNVTSVNTTGDREGLKNCSFNMTTELRDKRQKVYSLFYRLDIVPINENQGSEYRLINCNTSAITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQAFYATGDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIPGVITCKSDITGLLLLRDGGDTTDNTEIFRPGGGDMRDNWRSELYKYKVVEIKPLGVAPTDCKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICCTAVPWNISWSNKSKEEIWENMTWMQWDREINNYTNTIYRLLEESQNQQENNEKDLLALD

SEQ ID NO: 13 - protein, 641 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLDCSTYNNTHNISKEMKICSFNMTTELRDKKRKVNVLFYKLDLVPLTNSSNTTNYRLISCNTSTITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQSFYATGEIVGNIREAHCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPGGGDMRDNWRSELYKYKVVEIKPLGVAPTDCKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICCTAVPWNISWSNKSKEEIWENMTWMQWDREINNYTNTIYRLLEESQNQQENNEKDLLALD

SEQ ID NO: 14 - protein, 641 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLDCSTYNNTHNISKEMKICSFNMTTELRDKKRKVNVLFYKLDLVPLTNSSNTTNYRLISCNTSTITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQSFYATGEIVGNIREAHCNISGRNWSEAVNQVKKKLKEHFPHKNISFQSSSGGDLEITTHSFNCGGEFFYCNTSGLFNDTISNATIMLPCRIKQIINMWQEVGKAMYAPPIAGNITCKSDITGLLLLRDGGDTTDNTEIFRPGGGDMRDNWRSELYKYKVVEIKPLGVAPTDCKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICCTAVPWNISWSNKSKEEIWENMTWMQWDREINNYTNTIYRLLEESQNQQENNEKDLLALD

SEQ ID NO: 15 - protein, 643 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLQCTNYAPNLLSNMRGELKQCSFNMTTELRDKKRKVNVLFYKLDLVPLTNSSNTTNYRLISCNTSTITQACPKVTFDPIPIHYCAPAGYAILKCNNKTFNGKGPCNNVSTVQCTHGIKPVVSTQLLLNGSLAEEEIVIRSKDLSDNAKIIIVQLNTPVQINCTRPNNNTRKSIRIGPGQSFYATGEIIGDIRMAHCNVSKATWNEAVNQVKKKLKEHFPHKNISFQSSSGGDLEITTHSFNCGGEFFYCNTSGLFNDTISNATIVLPCRIKQIINMWQEVGKAMYAPPIQGVIRCVSNITGLLLLRDGGDTTDNTEIFRPGGGDMRDNWRSELYKYKVVEIKPLGVAPTDCKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICCTAVPWNISWSNKSKEEIWENMTWMQWDREINNYTNTIYRLLEESQNQQENNEKDLLALD

SEQ ID NO: 16 - protein, 643 aa, unidentified
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLHCTNVTPNLNTTGDREGLKNCSFNMTTELRDKRQKVYSLFYRLDIVPINENQGSEYRLINCNTSAITQACPKVTFDPIPIHYCAPAGYAILKCNNKTFNGKGPCNNVSTVQCTHGIKPVVSTQLLLNGSLAEEEIVIRSKDLSDNAKIIIVQLNKSVQINCTRPNNNTVKSIRIGPGQAFYYFGDIIGDIRMAYCNISGRNWSEAVNQVKKKLKEHFPHKNISFQSSSGGDLEITTHSFNCGGEFFYCNTSGLFNDTISNDSIMLPCRIKQIINMWQEVGKAIYAPPIPGVITCKSDITGLLLLRDGGDTTDNTEIFRPGGGDMRDNWRSELYKYKVVEIKPLGVAPTDCKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICCTAVPWNISWSNKSKEEIWENMTWMQWDREINNYTNTIYRLLEESQNQQENNEKDLLALD

SEQ ID NO: 17 - protein, 650 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWCQSLKPCVKLTPLCVTLHCTNVTISSTNGSTANVTMREEMKNCSFNTTTVIRDKIQKEYALFYKLDIVPIEGKNTNTGYRLINCNTSVITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPGNNTRRSINIGPGRAFYATGAIIGDIRQAYCNISGRNWSEAVNQVKKKLKEHFPHKNISFQSSSGGDLEITTHSFNCGGEFFYCNTSGLFNDTISNATIMLPCRIKQIINMWQEVGCGAIYAPPIKGNITCKSDITGLLLLRDGGDTTDNTEIFRPGGGDMRDNWRSELYKYKVVEIKPLGVAPTDAKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALD

SEQ ID NO: 18 - protein, 650 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWCQSLKPCVKLTPLCVTLHCTNVTISSTNGSTANVTMREEMKNCSFNTTTVIRDKIQKEYALFYKLDIVPIEGKNTNTGYRLINCNTSVTLQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPGNNTRRSINIGPGRAFYATGAIIGDIRQAYCNISGRNWSEAVNQVKKKLKEHFPHKNISFQSSSGGDLEITTHSFNCGGEFFYCNTSGLFNDTISNATIMLPCRIKQIINMWQEVGCGAIYAPPIKGNITCKSDITGLLLLRDGGDTTDNTEIFRPGGGDMRDNWRSELYKYKVVEIKPLGVAPTDAKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALD

SEQ ID NO: 19 - protein, 643 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWCQSLKPCVKLTPLCVTLHCTNVTSVNTTGDREGLKNCSFNMTTELRDKRQKVYSLFYRLDIVPINENQGSEYRLINCNTSAITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQAFYATGDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGCGAIYAPPIKGNITCKSDITGLLLLRDGGDTTDNTEIFRPGGGDMRDNWRSELYKYKVVEIKPLGVAPTDAKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALD

SEQ ID NO: 20 - protein, 643 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWCQSLKPCVKLTPLCVTLHCTNVTSVNTTGDREGLKNCSFNMTTELRDKRQKVYSLFYRLDIVPINENQGSEYRLINCNTSAITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQAFYATGDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGCGAIYAPPIPGVITCKSDITGLLLLRDGGDTTDNTEIFRPGGGDMRDNWRSELYKYKVVEIKPLGVAPTDAKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALD

SEQ ID NO: 21 - protein, 642 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWCQSLKPCVKLTPLCVTLDCSTYNNTHNISKEMKICSFNMTTELRDKKRKVNVLFYKLDLVPLTNSSNTTNYRLISCNTSTITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQSFYATGEIVGNIREAHCNISGRNWSEAVNQVKKKLKEHFPHKNISFQSSSGGDLEITTHSFNCGGEFFYCNTSGLFNDTISNATIMLPCRIKQIINMWQEVGCGAIYAPPIKGNITCKSDITGLLLLRDGGDTTDNTEIFRPGGGDMRDNWRSELYKYKVVEIKPLGVAPTDAKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALD

SEQ ID NO: 22 - protein, 642 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWCQSLKPCVKLTPLCVTLDCSTYNNTHNISKEMKICSFNMTTELRDKKRKVNVLFYKLDLVPLTNSSNTTNYRLISCNTSTITQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRKSIRIGPGQSFYATGEIVGNIREAHCNISGRNWSEAVNQVKKKLKEHFPHKNISFQSSSGGDLEITTHSFNCGGEFFYCNTSGLFNDTISNATIMLPCRIKQIINMWQEVGCGAMYAPPIAGNITCKSDITGLLLLRDGGDTTDNTEIFRPGGGDMRDNWRSELYKYKVVEIKPLGVAPTDAKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALD

SEQ ID NO: 23 - protein, 644 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWCQSLKPCVKLTPLCVTLQCTNYAPNLLSNMRGELKQCSFNMTTELRDKKRKVNVLFYKLDLVPLTNSSNTTNYRLISCNTSTITQACPKVTFDPIPIHYCAPAGYAILKCNNKTFNGKGPCNNVSTVQCTHGIKPVVSTQLLLNGSLAEEEIVIRSKDLSDNAKIIIVQLNTPVQINCTRPNNNTRKSIRIGPGQSFYATGEIIGDIRMAHCNVSKATWNEAVNQVKKKLKEHFPHKNISFQSSSGGDLEITTHSFNCGGEFFYCNTSGLFNDTISNATIVLPCRIKQIINMWQEVGCGAMYAPPIQGVIRCVSNITGLLLLRDGGDTTDNTEIFRPGGGDMRDNWRSELYKYKVVEIKPLGVAPTDAKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALD

SEQ ID NO: 24 - protein, 644 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWCQSLKPCVKLTPLCVTLHCTNVTPNLNTTGDREGLKNCSFNMTTELRDKRQKVYSLFYRLDIVPINENQGSEYRLINCNTSAITQACPKVTFDPIPIHYCAPAGYAILKCNNKTFNGKGPCNNVSTVQCTHGIKPVVSTQLLLNGSLAEEEIVIRSKDLSDNAKIIIVQLNKSVQINCTRPNNNTVKSIRIGPGQAFYYFGDIIGDIRMAYCNISGRNWSEAVNQVKKKLKEHFPHKNISFQSSSGGDLEITTHSFNCGGEFFYCNTSGLFNDTISNDSIMLPCRIKQIINMWQEVGCGAIYAPPIPGVITCKSDITGLLLLRDGGDTTDNTEIFRPGGGDMRDNWRSELYKYKVVEIKPLGVAPTDAKRRVVERRRRRRAVGIGAVFLGFLGAAGSTMGAASITLTVQARQLLSGIVQQQSNLLRAPEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALD

SEQ ID NO: 25 - protein, 734 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFNITTEIRDKTRKEYALFYRLDIVPLDNSSNPNSSNTYRLINCNTSTLTQACPKVTFDPIPIHYCAPAGYAILKCNNKTFNGKGPCNNVSTVQCTHGIKPVVSTQLLLNGSLAEEEIVIRSKNLSDNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGNTTNNTEIFRPGGGDMRDNWRSELYKYKVVEIKPLGVAPTDAKRRVVERNKRAVGIGAVFLGFLGAAGSTMGAASITLTVQARQLLSGIVQQQSNLLRAIEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALDSWNNLWNWFNITNWLWYIKIFIMIVGGLIGLRIIFIVLSIVNRVRQGYSPLSFQTLIPNPRGPDRPGGIEEEGGEQDRDRS

SEQ ID NO: 26 - protein, 734 aa, synthetic construct
MRVRGILRNWQQWWIWGILGFWMLMICSVVGNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFNITTEIRDKTRKEYALFYRLDIVPLDNSSNPNSSNTYRLINCNTSTLTQACPKVTFDPIPIHYCAPAGYAILKCNNKTFNGKGPCNNVSTVQCTHGIKPVVSTQLLLNGSLAEEEIVIRSKDLSDNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPGGGDMRDNWRSELYKYKVVEIKPLGVAPTDAKRRVVERNKRAVGIGAVFLGFLGAAGSTMGAASITLTVQARQLLSGIVQQQSNLLRAIEAQQHMLQLTVWGIKQLQTRVLAIERYLKDQQLLGLWGCSGKLICTTAVPWNISWSNKSKEEIWENMTWMQWDREINNYTNTIYRLLEESQNQQENNEKDLLALDSWNNLWNWFNITNWLWYIKIFIMIVGGLIGLRIIFIVLSIVNRVRQGYSPLSFQTLIPNPRGPDRPGGIEEEGGEQDRDRS

SEQ ID NO: 27 - protein, 849 aa, synthetic construct
MKVMGTKKNYQHLWRWGIMLLGMLMMSSAAEQLWVTVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREEMKNCSFNTTTVIRDKIQKEYALFYKLDIVPIEGKNTNTGYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIIGGLVGLRIVCAVLSIVNRVRQGYSPLSFQTRLPNPRGPDRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 28 - protein, 853 aa, synthetic construct
MRVMGIQRNCQHLLTWGIMILGTIIFCSAVENLWVTVYYGVPVWRDADTTLFCASDAKAYETEKHNVWATHACVPTDPNPQEIHLDNVTEKFNMWKNNMVEQMHTDIISLWDQSLKPCVKLTPLCVTLHCTNVTSVNTTGDREGLKNCSFNMTTELRDKRQKVYSLFYRLDIVPINENQGSEYRLINCNTSAITQACPKVSFEPIPIHYCTPAGFAILKCKDEGFNGTGLCKNVSTVQCTHGIKPVVSTQLLLNGSLAEKNITIRSENITNNAKIIIVQLVQPVTIKCIRPNNNTRKSIRIGPGQAFYATGDIIGDIRQAHCNVTRSRWNKTLQEVAEKLRTYFGNKTIIFANSSGGDLEITTHSFNCGGEFFYCNTSGLFNSTWYVNSTWNDTDSTQESNDTITLPCRIKQIINMWQRAGQAMYAPPIPGVIKCESNITGLLLTRDGGKDNNVNETFRPGGGDMRDNWRSELYKYKVVEIEPLGVAPTRAKRRVVEREKRAVGIGAVFLGFLGAAGSTMGAASITLTVQARQLLSGIVQQQNNLLRAIEAQQHLLKLTVWGIKQLQARVLAVERYLRDQQLLGIWGCSGKLICTTNVPWNSSWSNKSLDEIWNNMTWLQWDKEINNYTQLIYRLIEESQNQQEKNEKELLELDKWANLWSWFDISNWLWYIKIFIIIVGGLIGLRIVFAVLSVINRVRQGYSPLSFQTHTPNPRGLDRPERIEEEDGEQGRGRSIRLVSGFLALAWDDLRSLCLFSYHRLRDFILIAARTVELLGHSSLKGLRLGWEGIKYLWNLLSYWGRELKISAINLVDTIAIAVAGWTDRVIEIAQRIGRAILHIPVRIRQGLERALL

SEQ ID NO: 29 - protein, 835 aa, synthetic construct
MRVRGIMRNWQQWWIWGSLGFWMLIICNVMGSLWVTVYYGVPVWREAKTTLFCASDAKAYETEAHSVWATHACVPTDPNPQEMVLENVTENFNMWKNDMVDQMHEDVISIWDQSLKPCVKLTPLCVTLDCSTYNNTHNISKEMKICSFNMTTELRDKKRKVNVLFYKLDLVPLTNSSNTTNYRLISCNTSTITQACPKVSFDPIPIHYCAPAGYAILKCNNKTFNGTGPCNNVSTVQCTHGIKPVVSTQLLLNGSLAEEEIIIRFENLTDNVKIIIVQLNETINITCTRPNNNTRKSIRIGPGQSFYATGEIVGNIREAHCNISASKWNKTLERVRTKLKEHFPNKTIEFEPSSGGDLEITTHSFNCGGEFFYCNTSGLFNSAINGTLTSNVTLPCRIKQIINMWQEVGRAMYAPPIAGNITCKSNITGLLLTRDGGENSSSTTETFRPTGGDMKNNWRSELYKYKVVEIKPLGIAPTEAKRRVVEREKRAVGIGAVFLGFLGAAGSTMGAASMTLTVQARQLLSGIVQQQSNLLKAIEAQQHMLQLTVWGIKQLQARVLAIERYLKDQQLLGLWGCSGKLICTTNVPWNASWSNKSKNDIWDNMTWMQWDREISNHTDTIYRLLEDSQNQQEKNEKDLLALDSWKNLWNWFSITKWLWYIKIFIMIVGGLIGLRIIFAVLSIVNRVRQGYSPLSFQTLTPNPRGPDRLGGIEEEGGEQDKNKSRRLVTGFLPVVWDDLRSLCLFSYHLLRDFILIVARTVELLGRRGWEALKYLGGLVQYWGLELKKSTISLLDTIAIVVAEGTDRIIEVLQRIGRAIYNIPRRIRQGFETALL

(19) United States
(12) Patent Application Publication
(10) Pub. No.: US 2026/0248907 A1
(43) Pub. Date: Aug. 27, 2026

(54) DUAL-GERMLINE ANTIBODY ENGAGER HIV-1 ENVELOPE CHIMERIC IMMUNOGENS

(71) Applicant: The United States of America,as represented by the Secretary,Department of Health and Human Services, Bethesda, MD (US)

(72) Inventors: Peng Zhang, Rockville, MD (US); Paolo Lusso, Rockville, MD (US)

(73) Assignee: The United States of America,as represented by the Secretary,Department of Health and Human Services, Bethesda, MD (US)

(21) Appl. No.: 18/877,963

(22) PCT Filed: Aug. 11, 2023
(86) PCT No.: PCT/US2023/072123

§ 371 Date: Dec. 20, 2024

Related U.S. Application Data
(60) Provisional application No. 63/397,789, filed on Aug. 12, 2022.

(51) Int. Cl. A61K 39/21; A61K 39/00; A61K 39/395; A61P 37/04; C07K 14/005

(52) U.S. Cl. CPC A61K 39/21; A61K 39/39541; A61P 37/04; C07K 14/005; A61K 2039/545; A61K 2039/55516; A61K 2039/55522; A61K 2039/55527; A61K 2039/55533; A61K 2039/55538

(57) ABSTRACT

The invention provides an HIV-1 Env-derived immunogen that simultaneously engages more than one lineage of germline bNAbs specific for different neutralization target sites of the native HIV-1 Env trimer. In certain embodiments, the inventive immunogen comprises a chimeric Env, with binding sites drawn from more than one native Env proteins. These can be used both in the form of full-length/minimally truncated membrane-bound trimers (e.g., expressed from cDNA, mRNA or viral vectors, which also are provided by the present invention, as are cells comprising the immunogen) or, alternatively, in the form of soluble truncated trimers (e.g., SOSIP or IP trimers). Also provided are a pharmaceutical composition comprising the inventive immunogen, nucleic acids encoding the same, and/or cells comprising them, and a method of vaccinating a human patient against HIV using the inventive immunogen and composition.

### CROSS-REFERENCE TO RELATED APPLICATIONS

This patent application claims the benefit of U.S. Provisional Patent Application No. 63/397,789, filed Aug. 12, 2022, and which is incorporated by reference in its entirety.

### STATEMENT REGARDING FEDERALLY SPONSORED RESEARCH OR DEVELOPMENT

This invention was made with Government support under project numbers AI001314-03 and AI001197-09 by the National Institutes of Health, National Institute of Allergy and Infectious Diseases (NIAID). The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 633,638 Byte xml file named “767845.xml,” created on Aug. 11, 2023.

### BACKGROUND OF THE INVENTION

Despite four decades of intensive research, the development of a protective HIV vaccine has remained elusive due to the extreme difficulty in eliciting broadly neutralizing antibodies (bNAbs). For a summary of various approaches and the difficulties encountered, see Zhang et al., Nature Medicine, 27, 2234-45 (2021), which is incorporated herein in its entirety by reference. Such immunogens would be particularly useful when employed during the priming phase of an HIV vaccine regimen, involving a prime vaccine administration followed by one, or more typically several, booster vaccine administrations.

A first critical step in the process of eliciting bNABs is the activation and expansion of naïve B cells expressing unmutated germline antibodies of various bNAb lineages. Only rare natural HIV-1 envelopes (Envs) have the ability to engage such precursor B cells, but they do so invariably for a single lineage or for a few lineages specific for the same neutralization site (e.g., the CD4-binding site (CD4-BS)). Table 1 illustrates this, correlating specific native HIV-1 Envs that have been shown to engage precursor B cells to produce single-germline bNAbs, which are specific for but one target neutralization site on the HIV-1 Env, respectively:

TABLE 1
Only Rare Natural HIV-1 Envelopes Engage Germline bNAbs

| | | Target Neutralization |
|---|---|---|
| HIV-1 Env | Germline bNAb | Site |
| CH505 | CH103, CH235 | CD4-BS |
| 426c (including | VRC01, NIH45-46 | CD4-BS |
| deglyco1 and deglyco3) | | |
| CAP256 | VRC26 | V2 glycan |
| Q23.17 | CH01 | V2 glycan |
| WITO | CH01 | V2 glycan |
| A244 | CH01 | V2 glycan |
| ZM233 | CH01, PG9, VRC26 | V2 glycan |
| AG_250 | CH01, PG9, VRC26 | V2 glycan |

The sequences of these rare germline-engaging HIV-1 Env polypeptides (see Table 1, above) are known to those of skill in the art. Exemplary sequences of some of these are presented herein as follows: SEQ ID NO:25 (426c (WT.1-745)), SEQ ID NO:26 (426c (276D.460.463D.1-745)), SEQ ID NO:27 (WITO.33), SEQ ID NO:28 (Q23.17.gp160), and SEQ ID NO: 29 (ZM233). Additionally, sequence information concerning these Envs can be obtained from published sources (the following of which are incorporated herein in their entireties), such as:
For SH505, see doi.org/10.1038% 2Fnature12053 and doi.org/10.1126% 2Fscience.abd2638 (related to CAP256 as well),
For 426c, see doi.org/10.1038/ncomms10618, doi.org/10.1084/jem.20122824, and dx.doi.org/10.7554/eLife.37688;
For CAP256, see doi.org/10.1038/nature13036;
For Q23.17, see doi.org/10.1128/jvi.73.7.5255-5264.1999 and doi.org/10.1371/journal.ppat.1009807 (also related to WITO and T250);
For WITO, see doi.org/10.1128/jvi.79.16.10108-10125.2005;
For A244, see 90TH_CM244, doi.org/10.1128/jvi.79.10.6089-6101.2005, doi.org/10.1128/JVI.05045-11, and doi.org/10.1038% 2Fnsmb.3144 (related to WITO, T250, ZM233, Q23_17 and CAP256 as well);
For ZM233, see doi.org/10.1128/jvi.01730-06 and doi.org/10.1016/j.immuni.2015.10.014 (related to AG_250 as well);
For AG_250 and possibly T250-4, see doi.org/10.1128/jvi.00071-21.
   Additional cites can be obtained from the following table (see also Table 1 from Li et al., J. Virol. 95 (11), e00071-21 (2021), doi.org/10.1128/JVI.00071-21, which is incorporated herein in its entirety). Within this table, the HIV-1 Envs Q23.17, WITO4160, B41, CE1176, CH1012, ZM233, 1086, CH0694, RV217.40100, and T250-4 (as listed in the left column) are identified in Li et al. as “new SHIV constructions” and BG505, YU2, CH505, CH848, CAP256SU, and 191859 are identified in the reference as “Previous SHIV constructions”:

TABLE 2

| | | Env | SHIV | |
|---|---|---|---|---|
| | | GenBank | GenBank | |
| HIV-1 env | Subtype | accession no. | accession no. | Env properties |
| Q23.17 | A | AF004885 | MW410736 | Cloned from primary isolate; binds |
| | | | | V1V2 bNAb UCAs |
| WITO4160 | B | FJ496176 | MW410737 | T/F^{a}; binds V1V2 bNAb UCAs |
| B41^{b} | B | EU576114 | MW410732 | T/F; SOSIP immunogen |
| CE1176 | C | FJ444437 | MW410733 | T/F; global neutralization panel |
| CH1012 | C | MG898887 | MW410734 | T/F; elicited bNAbs in human |
| ZM233 | C | DQ388517 | MW410738 | Cloned from primary isolate; binds |
| | | | | V1V2 bNAb UCAs |
| 1086 | C | FJ444395 | MW410739 | T/F; P5 vaccine trial |
| CH0694 | C | KJ700458 | MW410741 | T/F; elicited bNAbs in human |
| RV217.40100 | AE | MN792078 | MW410740 | T/F; Thai AE subtype |
| T250-4^{c} | AG | MW507842 | MW410735 | Primary isolate; binds |
| | | | | V1V2 bNAb UCAs |
| BG505^{d} | A | DQ208458 | KU958484 | T/F; elicited bNAbs in human; |
| | | | | SOSIP immunogen |
| YU2 | B | M93258 | KU958489 | Macrophage-tropic; brain-derived |
| CH505 | C | KC247556 | KU958487 | T/F; elicited bNAbs in human |
| CH848 | C | KX216883 | KU958488 | T/F; elicited bNAbs in human |
| CAP256SU | C | KF241776 | MT509359 | T/F; binds V1V2 bNAb UCAs |
| 191859 | D | JX203061 | KU958486 | T/F; macrophage-tropic |

Researchers have also engineered synthetic gp120 core molecules or peptides that selectively engage and activate a single lineage, such as the eOD-GT8 protein for the CD4-binding site antibody VRC01 and related bNAbs. Also, some authors have reported HIV-1 Envs that can engage more than 1 bNAb lineage, most of these studies reported closely related lineages targeting the same antigenic site (most often the CD4 BS or the V2 glycan region) (Stamatatos et al., Immunol. Rev. 275, 203-216 (2017), which is incorporated herein in its entirety). Furthermore, Medina-Ramirez et al. (J. Exp. Med. 214, 2573-2590 (2017), which is incorporated herein in its entirety) constructed a mutated BG505 SOSIP trimer variant, named SOSIP.v4.1-GT1, containing 18 mutations (nine substitutions in V2 (K169R, Y173H, S174A, R178K, V181I, Q183P, G188N, N189T, and E190S), a seven-residue deletion in V2, six sequence changes around the CD4bs (S199A, N276D, T278R, N386D, N462D, and T471S), as well as the E64K and A316W substitutions), which appears to engage simultaneously germline antibodies against the CD4-BS and the V2 glycan region. However, it is unclear whether the germline-reverted antibodies used in Medina-Ramirez et al. were really close to the germline sequences, especially for PG9, PG16 and CH01, since these putative germline antibodies bound with comparable affinity to the parental (unmodified) SOSIP.v4.1 SOSIP trimer. Likewise, Escolano et al. (Nature 570, 468-473 (2019), which is incorporated herein in its entirety) have heavily modified the BG505 SOSIP trimer to generate a variant, RC1, that engages inferred germline antibodies directed against the V3-glycan patch, such as germline PGT121.

Thus, what is needed, and has remained elusive, is the generation of a single Env immunogen capable of simultaneously engaging multiple germline bNAb lineages. The present invention addresses this unmet clinical need.

### BRIEF SUMMARY OF THE INVENTION

The invention provides an HIV-1 Env-derived immunogen that simultaneously engages more than one lineage of germline bNAbs specific for different neutralization target sites of the native HIV-1 Env trimer. In certain embodiments, the inventive immunogen comprises a chimeric Env, with binding sites drawn from more than one native Env proteins. These can be used both in the form of full-length/minimally truncated membrane-bound trimers (e.g., expressed from cDNA, mRNA, or viral vectors, which also are provided by the present invention, as are cells comprising the immunogen) or, alternatively, in the form of soluble truncated trimers (e.g., SOSIP or IP trimers). Also provided are a pharmaceutical composition comprising the inventive immunogen, nucleic acids encoding the same, and/or cells comprising them, and a method of method of vaccinating a human patient against HIV using the inventive immunogen and composition.

This invention provides a much-needed advancement in the field because it provides optimized immunogens to be employed especially during the priming phase of an HIV vaccine regimen. This invention represents a key breakthrough that provides a new generation of germline-engaging Env that are inherently superior to single germline engagers for HIV-1 vaccine priming.

### BRIEF DESCRIPTION OF THE SEVERAL VIEWS OF THE DRAWING(S)

FIG. 1 schematically depicts the design of a dual germline engager chimeric Env immunogen according to the present invention, in which the V1-V2-V3 loop regions from a first source native HIV-1 Env polypeptide (426c, which, according to Table 1, engages VRC01, targeting CD4-BS) is replaced with the V1-V2-V3 loop regions from a second source native HIV-1 Env polypeptide (WITO, which, according to Table 1, engages CH01, targeting the V2 glycan).

FIG. 2 is an amino acid sequence alignment pertaining to the design of dual germline-engager Envs relating to WITO and Q23. The sequences are as indicated. Boxes represent the sequences that were exchanged between the 2 strains to generate the chimera.

FIG. 3 is an amino acid sequence alignment pertaining to the design of dual germline-engager Envs relating to Q23. The sequences are as indicated. Boxes represent the sequences that were exchanged between the 2 strains to generate the chimera.

FIG. 4 is an amino acid sequence alignment pertaining to the design of dual germline-engager Envs relating to ZM233. The sequences are as indicated. Boxes represent the sequences that were exchanged between the 2 strains to generate the chimera.

FIG. 5 is a combined image representing the design of a dual germline engager chimeric Env immunogen according to the present invention, in which the V1-V2-V3 loop regions from a first source native HIV-1 Env polypeptide (426c deglyco-3 variant of 426c, which, according to Table 1, engages VRC01, targeting CD4-BS) is replaced with the V1-V2-V3 loop regions from a second source native HIV-1 Env polypeptide (WITO.27 variant of WITO, which, according to Table 1, engages CH01, targeting the V2 glycan). Superimposed with the schematic diagrams are bar charts representing data comparing the antigenicity of the two “source” native HIV-1 Env polypeptides with the chimeric molecule.

FIG. 6 is a bar chart presenting data concerning the antigenic profiles of wild-type and dual germline engager chimeric Env immunogens according to the present invention.

FIG. 7 is a bar chart presenting data concerning the antigenic profiles of wild-type and dual germline engager chimeric Env immunogens according to the present invention. In the figure legend, “WITO chimera” refers to 426c_276D_460D463D_WITO_V12V3 (SEQ ID NO: 1), and “Q23 chimera” refers to 426c_276D_460D463D_Q23_V123 (SEQ ID NO:3).

FIG. 8 schematically represents four arms of a clinical trial comparing a dual germline engager chimeric Env immunogen according to the present invention (Arm 4), as a prime dose in the immunization regimen, against three other prime immunogens. In the figure, “Gag” refers to the Gag gene of HIV-1 and “BG+REJO” refers to a mixture of the BG505 and the REJO Env genes.

FIG. 9 schematically represents four arms of a study in Macaques comparing a dual germline engager chimeric Env immunogen according to the present invention (Arm 4), as a prime dose in the immunization regimen, against three other prime immunogens. In the figure, the Total mRNA dose column represents HIV-1 Env, Gag, and GagPol mRNA in a ratio of 1.5:1:0.1 ratio (150 μg:100 μg:10 μg). For Arm 1, 426c-ΔG3 is employed in a classic prime (cp) regimen. For Arm 2, the 426c-Q23 chimera is employed in a cp regimen. For Arm 3, the 426c-Q23 chimera also is employed in a cp regimen. For Arm 4, 426c-Q23 chimera is employed in a dose-escalation/long-interval regimen according to which, during month 0, dosage of 426c-Q23 chimera is repeated at escalating dosage as indicated with no dosing at month 2.

FIG. 10 presents data collected from Macaques immunized in accordance with the dosing regimen depicted in FIG. 9. The assay involved binding to BG505 SOSIP.664 trimer. Group 1 represents Arm 1 from FIG. 9; Group 2 represents Arm 2 from FIG. 9; Group 3 represents Arm 3 from FIG. 9; and Group 4 represents Arm 4 from FIG. 9.

FIG. 11 presents data collected from Macaques 18 weeks following immunization in accordance with the dosing regimen depicted in FIG. 9. The assay involved the neutralization of a tier-1B heterologous (i.e., not 426c) strain: BaL. The four columns of data in the graph represent the four Arms as depicted in FIG. 9

FIG. 12 presents data collected from Macaques 18 weeks following immunization in accordance with the dosing regimen depicted in FIG. 9. The assay involved the neutralization of a tier-2 homologous strain that is difficult to neutralize: wild-type 426c. The four columns of data in the graph represent the four Arms as depicted in FIG. 9.

FIG. 13 presents data collected from Macaques 18 weeks following immunization in accordance with the dosing regimen depicted in FIG. 9. The assay involved the neutralization of a tier-2 heterologous strain that is difficult to neutralize: AD8. The four columns of data in the graph represent the four Arms as depicted in FIG. 9.

### DETAILED DESCRIPTION OF THE INVENTION

In one aspect, the invention provides an HIV-1 Env-derived immunogen that simultaneously engages more than one germline lineage of broadly neutralizing antibodies (bNAbs), wherein each of the bNABs exhibits specificity for a distinct neutralization site of the HIV-1 envelope. As noted in Table 1, exemplary distinct neutralization sites include the CD4-binding site (CD4-BS) or the V2 glycan supersite of the HIV-1 envelope. Additional neutralization sites include the V3 glycan supersite, the gp120/gp41 interface, the fusion peptide region, and the MPER domain of gp41. Thus, particularly when one of the germline lineages of bNAbs engaged by the inventive immunogen targets the CD4-BS, such bNAb can be N6, CH103, VRC01, 3BNC117, or another germline bNAb targeting CD4-BS of the HIV-1 envelope protein (such as CH235 and NIH45-46, as identified in Table 1). Similarly, and particularly when one of the germline lineages of bNAbs engaged by the inventive immunogen comprises the V2 glycan of the HIV-1 envelope, such bNAb can be CH01, PG9, PG16, PGT145, VRC26, or another germline bNAb targeting the V2 glycan of the HIV-1 envelope protein. Also, and particularly when one of the germline lineages of bNAbs engaged by the inventive immunogen comprises the V3 glycan of the HIV-1 envelope, such bNAb can be PGT121, PGT128, 10-1074, or another germline bNAb targeting the V3 glycan of the HIV-1 envelope protein.

The invention achieves providing an HIV-1 Env-derived immunogen that simultaneously engages more than one germline lineage of bNAbs by providing a chimeric Env-derived protein (polypeptide) and associated mRNA, expression vector, cells comprising the same, and pharmaceutical composition suitable for immunizing a human patient.

Thus, in one aspect, the invention provides a chimeric polypeptide comprising at least two of (a) a first polyamino acid domain representing the “core” structure of a first native HIV-1 Env polypeptide that engages N6, CH103, VRC01, 3BNC117, and/or NIH45-46 germline bNABs or those targeting CD4-BS, (b) a second polyamino acid domain representing the V1-V2-V3 loop regions of a second native HIV-1 Env polypeptide that engages CH01, PG9, PGT145 and/or VRC26 germline bNABs or those targeting the V2 glycan, and or (c) a third polyamino acid domain that engages PGT121, PGT128, or 10-1074 germline bNABs targeting the V3 supersite of the HIV-1 envelope protein.

Thus, for example, the first polyamino acid of the inventive chimeric immunogen can comprise the core domain from an HIV-1 envelope polypeptide such as CH505 and 426c, and the second polyamino acid of the inventive chimeric immunogen can comprise the V1-V2-V3 loop regions from an HIV-1 envelope polypeptide such as CAP256, Q23.17, WITO, A244, ZM233, and AG_250. It will be observed that, when variants of these native HIV-1 Env proteins/polypeptides are known or discovered, such can be employed in the context of the present invention as well. Thus, for example, in certain preferred embodiments, the 426c core is that of the 426c-deglyco3 (N267D/N460D/N463D) variant, which exposes the CD4BS in a more open fashion. Similarly, in certain preferred embodiments, the Q23.17V1-V2-V3 loop regions are those of the Q23.17 variant.

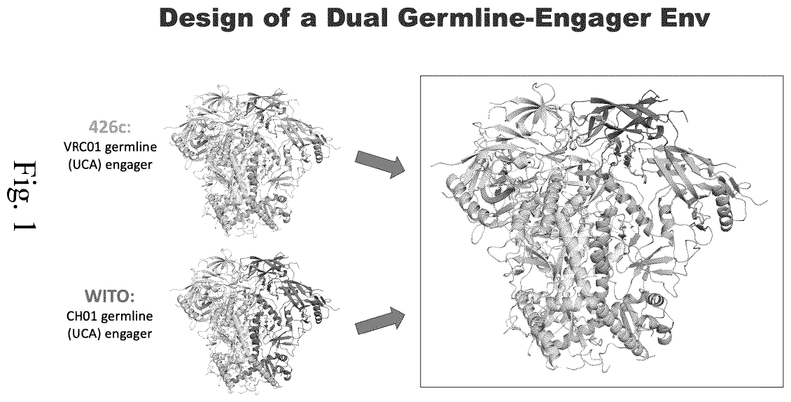

To construct the inventive chimeric immunogen, the genetic sequence encoding the V1-V2-V3 loop regions of the first “source” native HIV-1 Env polypeptide that that engages CH103, CH235, VRC01, and/or NIH45-46 germline bNABs or those targeting CD4-BS is replaced with the V1-V2-V3 loop regions of the second “source” native HIV-1 Env polypeptide that engages CH01, PG9, and/or VRC26 germline bNABs or those targeting the V2 glycan. This is schematically illustrated in FIG. 1. Additionally or alternatively, either or both of the first and second “source” sequences can be augmented with the addition of a third polyamino acid domain that engages germline bNABs targeting the V3 supersite of the HIV-1 envelope protein.

In addition to replacing the V1-V2-V3 loop regions of the first native source HIV-1 Env polypeptide with that of the second and/or augmenting either with the third native HIV-1 Env polypeptide that engages germline bNABs targeting the V3 supersite, rational design can be undertaken to further mutate the sequence so as to produce a resulting chimeric polypeptide that properly folds and trimerizes. Proper folding/trimerization then can be assessed by expressing the putative chimeric molecule and assaying for proper trimerization, for example using flow cytometry assay using standard methodology.

It will be observed that the resulting chimeric molecule, encoded by the engineered genetic sequence, retains the capacity of the first native source HIV-1 Env polypeptide to engage the CH103, CH235, VRC01, and/or NIH45-46 germline bNABs or those targeting the CD4-BS and also possesses the capacity of the second native source HIV-1 Env polypeptide to engage the CH01, PG9, and/or VRC26 germline bNABs or those targeting the V2 glycan and/or the capacity of the third native source NIH-1 Env polypeptide to engage bNAbs targeting the V3 supersite of the HIV-1 Env protein, or other neutralization sites, such as the gp120/gp41 interface, the fusion peptide region, and the MPER domain of gp41.

In certain embodiments, the inventive HIV-1 Env-derived immunogen is presented as a protein/polypeptide. Such can be a membrane-bound protein/polypeptide or a soluble protein/polypeptide, and the invention provides embodiments of each. Non-limiting examples of membrane-bound chimeric proteinaceous HIV-1 Env-derived immunogens of the present invention are represented by SEQ ID NOs: 1-8. In particular:

SEQ ID NO:1 represents an embodiment in which the first source native HIV-1 Env polypeptide is the 426c-deglyco3 (N267D/N460D/N463D) variant and the second source HIV-1 Env polypeptide is WITO. Thus, for SEQ ID NO:1, the V1-V2-V3 loop regions of 426c-deglyco3 (N267D/N460D/N463D) have been replaced with those from WITO.

SEQ ID NO:2 represents an embodiment in which the first source native HIV-1 Env polypeptide is the 426c-deglyco3 (N267D/N460D/N463D) variant and the second source HIV-1 Env polypeptide is WITO. Thus, for SEQ ID NO: 1, the V1-V2-V3 loop regions of 426c-deglyco3 (N267D/N460D/N463D) have been replaced with those from WITO and a TL substitution added. “TL” indicates additional amino acid replacements introduced to optimize the interface between the graft and the host molecule. TL corresponds to positions 200 and 201 (originally VI in WITO but changed to TL in 426c).

SEQ ID NO:3 represents an embodiment in which the first source native HIV-1 Env polypeptide is the 426c-deglyco3 (N267D/N460D/N463D) variant and the second source HIV-1 Env polypeptide is Q23. Thus, for SEQ ID NO:3, the V1-V2-V3 loop regions of 426c-deglyco3 (N267D/N460D/N463D) have been replaced with those from Q23.17.

SEQ ID NO:4 represents an embodiment in which the first source native HIV-1 Env polypeptide is the 426c-deglyco3 (N267D/N460D/N463D) variant and the second source HIV-1 Env polypeptide is Q23. Thus, for SEQ ID NO:4, the V1-V2-V3 loop regions of 426c-deglyco3 (N267D/N460D/N463D) have been replaced with those from Q23.17 and a PGV substitution added. “PGV” indicates additional amino acid replacements introduced to optimize the interface between the graft and the host molecule. PGV corresponds to positions 440-442 (originally KGN in 426c but changed to PGV in Q23.17)

SEQ ID NO:5 represents an embodiment in which the first source native HIV-1 Env polypeptide is the 426c-deglyco3 (N267D/N460D/N463D) variant and the second source HIV-1 Env polypeptide is ZM233. Thus, for SEQ ID NO:5, the V1-V2-V3 loop regions of 426c-deglyco3 (N267D/N460D/N463D) have been replaced with those from ZM233.

SEQ ID NO:6 represents an embodiment in which the first source native HIV-1 Env polypeptide is the 426c-deglyco3 (N267D/N460D/N463D) variant and the second source HIV-1 Env polypeptide is ZM233. Thus, for SEQ ID NO:6, the V1-V2-V3 loop regions of 426c-deglyco3 (N267D/N460D/N463D) have been replaced with those from ZM233 and an MA substitution added. “MA” indicates additional amino acid replacements introduced to optimize the interface between the graft and the host molecule. MA corresponds to positions 328 and 329 (originally QA in 426c but changed to MA in ZM233).

SEQ ID NO:7 represents an embodiment in which the first source native HIV-1 Env polypeptide is 426c and the second source HIV-1 Env polypeptide is ZM233. Thus, for SEQ ID NO: 7, the V1-V2-V3 loop regions of 426c have been replaced with those from ZM233. Additionally, the chimera represented by SEQ ID NO:7 has been engineered to engage germline bNAbs against a third vulnerability site of the HIV-1 envelope: the V3 glycan supersite.

SEQ ID NO:8 represents an embodiment in which the first source native HIV-1 Env polypeptide is 426c and the second source HIV-1 Env polypeptide is Q23.17. Thus, for SEQ ID NO: 8, the V1-V2-V3 loop regions of 426c have been replaced with those from Q23.17. Additionally, the chimera represented by SEQ ID NO:8 has been engineered to engage germline bNAbs against a third vulnerability site of the HIV-1 envelope: the V3 glycan supersite.

Embodiments in which the chimeric proteinaceous HIV-1 Env-derived immunogens of the present invention are membrane-bound, such as SEQ ID NOs: 1-8, such can be presented within the membrane of a living cell, or a ghost or corpuscle. Typically, a membrane-bound chimeric proteinaceous HIV-1 Env-derived immunogen of the present invention is contained within the cell membrane of a living cell. Thus, in an embodiment, the invention provides a cell comprising a membrane-bound chimeric proteinaceous HIV-1 Env-derived immunogens of the present invention. The cell can be any desired cell, and typically is a human cell suitable for use as an agent in a cell-based vaccine composition suitable for administration to human patients. Thus, desirably, the cell is not a transformed (cancerous) cell. Examples of suitable cells for use in the context of the present invention for presenting the inventive chimeric proteinaceous HIV-1 Env-derived immunogen include but are not limited to primary dendritic cells derived from the vaccine recipient or an HLA-compatible donor.

As an alternative to membrane-bound embodiments, a chimeric proteinaceous HIV-1 Env-derived immunogen of the present invention can instead be a soluble protein/polypeptide. For example, such can be produced as a soluble IP or SOSIP trimer. Non-limiting examples of soluble chimeric proteinaceous HIV-1 Env-derived immunogens of the present invention are represented by SEQ ID NOs: 9-24. In particular:

SEQ ID NO:9 represents an embodiment in which the first source native HIV-1 Env polypeptide is the 426c-deglyco3 (N267D/N460D/N463D) variant and the second source HIV-1 Env polypeptide is WITO. Thus, for SEQ ID NO:9, the V1-V2-V3 loop regions of 426c-deglyco3 (N267D/N460D/N463D) have been replaced with those from WITO. The sequence has been further derivatized to produce the polypeptide as a SOSIP trimer.

SEQ ID NO:10 represents an embodiment in which the first source native HIV-1 Env polypeptide is the 426c-deglyco3 (N267D/N460D/N463D) variant and the second source HIV-1 Env polypeptide is WITO. Thus, for SEQ ID NO:10, the V1-V2-V3 loop regions and TL of 426c-deglyco3 (N267D/N460D/N463D) have been replaced with those from WITO. The TL substitution is discussed above with reference to SEQ ID NO:2. The sequence has been further derivatized to produce the polypeptide as a SOSIP trimer.

SEQ ID NO:11 represents an embodiment in which the first source native HIV-1 Env polypeptide is the 426c-deglyco3 (N267D/N460D/N463D) variant and the second source HIV-1 Env polypeptide is Q23. Thus, for SEQ ID NO:11, the V1-V2-V3 loop regions of 426c-deglyco3 (N267D/N460D/N463D) have been replaced with those from Q23. The sequence has been further derivatized to produce the polypeptide as a SOSIP trimer.

SEQ ID NO:12 represents an embodiment in which the first source native HIV-1 Env polypeptide is the 426c-deglyco3 (N267D/N460D/N463D) variant and the second source HIV-1 Env polypeptide is Q23. Thus, for SEQ ID NO: 12, the V1-V2-V3 loop regions of 426c-deglyco3 (N267D/N460D/N463D) have been replaced with those from Q23 and a PGV substitution added. The PGV substitution is discussed above with reference to SEQ ID NO:4. The sequence has been further derivatized to produce the polypeptide as a SOSIP trimer.

SEQ ID NO:13 represents an embodiment in which the first source native HIV-1 Env polypeptide is the 426c-deglyco3 (N267D/N460D/N463D) variant and the second source HIV-1 Env polypeptide is ZM233. Thus, for SEQ ID NO:13, the V1-V2-V3 loop regions of 426c-deglyco3 (N267D/N460D/N463D) have been replaced with those from ZM233. The sequence has been further derivatized to produce the polypeptide as a SOSIP trimer.

SEQ ID NO:14 represents an embodiment in which the first source native HIV-1 Env polypeptide is the 426c-deglyco3 (N267D/N460D/N463D) variant and the second source HIV-1 Env polypeptide is ZM233. Thus, for SEQ ID NO:14, the V1-V2-V3 loop regions of 426c-deglyco3 (N267D/N460D/N463D) have been replaced with those from ZM233 and an MA substitution added. The MA substitution is discussed above with reference to SEQ ID NO:6. The sequence has been further derivatized to produce the polypeptide as a SOSIP trimer.

SEQ ID NO:15 represents an embodiment in which the first source native HIV-1 Env polypeptide is 426c and the second source HIV-1 Env polypeptide is ZM233. Thus, for SEQ ID NO:15, the V1-V2-V3 loop regions of 426c have been replaced with those from ZM233. Additionally, the chimera represented by SEQ ID NO:15 has been engineered to engage germline bNAbs against a third vulnerability site of the HIV-1 envelope: the V3 glycan supersite. The sequence has been further derivatized to produce the polypeptide as a SOSIP trimer.

SEQ ID NO:16 represents an embodiment in which the first source native HIV-1 Env polypeptide is 426c and the second source HIV-1 Env polypeptide is Q23.17. Thus, for SEQ ID NO:16, the V1-V2-V3 loop regions of 426c have been replaced with those from Q23. Additionally, the chimera represented by SEQ ID NO:16 has been engineered to engage germline bNAbs against a third vulnerability site of the HIV-1 envelope: the V3 glycan supersite. The sequence has been further derivatized to produce the polypeptide as a SOSIP trimer.

SEQ ID NO:17 represents an embodiment in which the first source native HIV-1 Env polypeptide is the 426c-deglyco3 (N267D/N460D/N463D) variant and the second source HIV-1 Env polypeptide is WITO. Thus, for SEQ ID NO:17, the V1-V2-V3 loop regions of 426c-deglyco3 (N267D/N460D/N463D) have been replaced with those from WITO. The sequence has been further derivatized to produce the polypeptide as an IP trimer.

SEQ ID NO:18 represents an embodiment in which the first source native HIV-1 Env polypeptide is the 426c-deglyco3 (N267D/N460D/N463D) variant and the second source HIV-1 Env polypeptide is WITO. Thus, for SEQ ID NO:18, the V1-V2-V3 loop regions and TL of 426c-deglyco3 (N267D/N460D/N463D) have been replaced with those from WITO. The TL substitution is discussed above with reference to SEQ ID NO:2. The sequence has been further derivatized to produce the polypeptide as an IP trimer.

SEQ ID NO:19 represents an embodiment in which the first source native HIV-1 Env polypeptide is the 426c-deglyco3 (N267D/N460D/N463D) variant and the second source HIV-1 Env polypeptide is Q23. Thus, for SEQ ID NO: 19, the V1-V2-V3 loop regions of 426c-deglyco3 (N267D/N460D/N463D) have been replaced with those from Q23. The sequence has been further derivatized to produce the polypeptide as an IP trimer.

SEQ ID NO:20 represents an embodiment in which the first source native HIV-1 Env polypeptide is the 426c-deglyco3 (N267D/N460D/N463D) variant and the second source HIV-1 Env polypeptide is Q23. Thus, for SEQ ID NO:20, the V1-V2-V3 loop regions of 426c-deglyco3 (N267D/N460D/N463D) have been replaced with those from Q23 and a PGV substitution added. The PGV substitution is discussed above with reference to SEQ ID NO:4. The sequence has been further derivatized to produce the polypeptide as an IP trimer.

SEQ ID NO:21 represents an embodiment in which the first source native HIV-1 Env polypeptide is the 426c-deglyco3 (N267D/N460D/N463D) variant and the second source HIV-1 Env polypeptide is ZM233. Thus, for SEQ ID NO:21, the V1-V2-V3 loop regions of 426c-deglyco3 (N267D/N460D/N463D) have been replaced with those from ZM233. The sequence has been further derivatized to produce the polypeptide as an IP trimer.

SEQ ID NO:22 represents an embodiment in which the first source native HIV-1 Env polypeptide is the 426c-deglyco3 (N267D/N460D/N463D) variant and the second source HIV-1 Env polypeptide is ZM233. Thus, for SEQ ID NO:22, the V1-V2-V3 loop regions of 426c-deglyco3 (N267D/N460D/N463D) have been replaced with those from ZM233 and an MA added. The MA substitution is discussed above with reference to SEQ ID NO:6. The sequence has been further derivatized to produce the polypeptide as an IP trimer.

SEQ ID NO:23 represents an embodiment in which the first source native HIV-1 Env polypeptide is 426c and the second source HIV-1 Env polypeptide is ZM233. Thus, for SEQ ID NO:23, the V1-V2-V3 loop regions of 426c have been replaced with those from ZM233. Additionally, the chimera represented by SEQ ID NO:23 has been engineered to engage germline bNAbs against a third vulnerability site of the HIV-1 envelope: the V3 glycan supersite. The sequence has been further derivatized to produce the polypeptide as an IP trimer.

SEQ ID NO:24 represents an embodiment in which the first source native HIV-1 Env polypeptide is 426c and the second source HIV-1 Env polypeptide is Q23.17. Thus, for SEQ ID NO:24, the V1-V2-V3 loop regions of 426c have been replaced with those from Q23.17. Additionally, the chimera represented by SEQ ID NO:24 has been engineered to engage germline bNAbs against a third vulnerability site of the HIV-1 envelope: the V3 glycan supersite. The sequence has been further derivatized to produce the polypeptide as an IP trimer.

Also encompassed in the present invention are functional variants of the specific exemplary immunogens, the sequences of which differ from those presented in SEQ ID NOs: 1-24. With regard to such variants, any type of alteration in the amino acid sequence is permissible so long as the variant retains the immunogenic activity described herein and is able to trimerize appropriately as an HIV-1 Env trimer. Examples of such variations include, but are not limited to, amino acid deletions, amino acid insertions, amino acid substitutions and combinations thereof. For example, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein.

As noted, isolated variant proteins of the present invention can also contain amino acid substitutions as compared to the exemplary sequences disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions.

Desired amino acid substitutions (whether conservative or non-conservative) can be determined by those skilled in the art at the time such substitutions are desired. For example, in one aspect, the inventive immunogen comprises at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or any ranges of values thereof) amino acid substitution (e.g., conservative substitution). In one aspect, the inventive immunogen protein comprises an amino acid sequence that is at least 90%, at least 91%, at least 92%, at least 93%, at least 94%, at least 95%, at least 96%, at least 97%, at least 98%, or at least 99% identical to one of the specific sequences (SEQ ID NO:s 1-24) described herein.

In embodiments in which the inventive chimeric proteinaceous HIV-1 Env-derived immunogen of the present invention comprises a soluble protein/polypeptide, such can be present in solution (e.g., a composition, such as a pharmaceutical composition as described herein). Alternatively, such soluble polypeptide or protein can be isolated and prepared for storage and/or shipping, for example in lyophilized form. In such embodiments, the inventive polypeptide can be presented in the presence of agents, such as lyoprotectants (e.g., trehalose), if desired.

Inasmuch as the inventive Env-derived immunogen can be proteinaceous (e.g., a protein or polypeptide), in an embodiment, the invention provides a nucleic acid molecule encoding the HIV-1 Env-derived immunogen of the present invention. Such nucleic acid can be DNA (e.g., cDNA) or RNA (e.g., mRNA). The nucleic acid can be employed within a cell ex vivo to produce the inventive Env-derived immunogen either as a membrane-bound protein, as described, or in soluble form. Alternatively, the nucleic acid can be employed in vivo to produce the inventive Env-derived immunogen within a host (e.g., a human patient) to achieve immunization against HIV.

Additionally, such nucleic acid can be included within a gene transfer and/or expression vector for expressing the inventive Env-derived immunogen as a protein within a cell. Any suitable vector system can be employed in the context of the present invention. For example, the vector can be an adenovirus vector, an adeno-associated virus (AAV) vector, a poxvirus vector, a plasmid, or any other desired vector suitable for expressing the nucleic acid within the desired cell type. A person of ordinary skill is enabled to select an appropriate genetic vector for expressing the nucleic acid encoding the inventive Env-derived immunogen in the context of the present invention.

In an embodiment, the invention further provides a pharmaceutical composition comprising (a) an HIV-1 Env-derived immunogen of the present invention, including a cell comprising such, for example involving embodiments in which the immunogen is membrane-bound, (b) a nucleic acid molecule encoding an inventive HIV-1 Env-derived immunogen, as described herein, (c) a genetic vector comprising such a nucleic acid, alone or in combination (as “active compound(s)”), and a pharmaceutically acceptable carrier.

The inventive composition can be formulated for administration to a human patient by any desired route, such as via injection (e.g., intravenously, intramuscularly, or otherwise), or via aerosol (e.g., via inhalation, intranasally, or otherwise), by the oral route, by topical application to the genital mucosa, or by any other desired mode of administration. Typically, however, the inventive pharmaceutical composition will be formulated for injection and will comprise, as the bulk of the carrier, physiologically-compatible saline, suitable buffers, preservatives, and other excipients known to those of ordinary skill in the art of pharmaceutical formulation.

Within the composition, the active compound(s) can be present at any desired concentration or dosage. For example, where the active compound(s) is a protein or polypeptide (such as the soluble IP or SOSIP trimers described herein), such can be present in the inventive composition in an amount between 10 μg and 1000 μg, such as at least 20 μg, or at least 50 μg, or at least 100 μg, such as 250 μg or more, or even 500 μg or higher amounts up to 1000 μg. Indeed, if desired, even a greater amount of the HIV-1 Env-derived immunogen of the present invention can be present in the formulation of the inventive pharmaceutical composition. Similarly, when the pharmaceutical composition comprises cells (such as those carrying the inventive HIV-1 Env-derived immunogen as a membrane-bound protein/polypeptide, such can be present in a concentration of 105 to 10¹² cells/ml, such as at least 10⁶ cells/ml, or at least 10⁷ cells/ml, or at least 10⁸ cells/ml, or at least 10⁹ cells/ml, or at least 10¹¹ cells/ml.

The carrier portion of the pharmaceutical composition of the present invention can be any of those conventionally used and is limited only by physio-chemical considerations, such as solubility and lack of reactivity with the active compound(s), and by the route of administration. The choice of carrier will be determined in part by the particular peptide, polypeptide, nucleic acid, vector, cell, or composition thereof of the invention and other active agents or drugs used, as well as by the particular method used to administer the peptide, polypeptide, nucleic acid, vector, cell, or composition thereof. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The HIV-1 gag gene, alone or together with the protease gene, preferably administered in the form of full-length or truncated gagpol transcript, can be added in order to promote the in vivo formation of virus-like particles (VLPs), which are more effective immunogens than the Env protein alone.

Adjuvants such as alum, CpG, ADJUPLEX, chitosan, and the like also can be included in the inventive pharmaceutical; composition, as desired, to aid in formulation and administration.

The composition additionally or alternatively can comprise one or more immunostimulatory/regulatory molecules. Any suitable immunostimulatory/regulatory molecule can be used, such as interleukin (IL)-2, IL-4, IL-6, IL-7, IL-12, IL-15, IL-15/IL-15Ra, IL-15/IL-15Ra-Fc, interferon (IFN)-γ, tumor necrosis factor (TNF)-α, B7.1, B7.2, ICAM-1, ICAM-2, LFA-1, LFA-2, LFA-3, CD70, CD-72, RANTES, G-CSF, GM-CSF, OX-40L, 41 BBL, anti-CTLA-4, IDO inhibitor, anti-PDL1, anti-PD1, and combinations thereof. In one embodiment, the IL-12 is NHS-IL12, which is an immunocytokine composed of two IL-12 heterodimers fused to the NHS76 antibody (see Strauss et al., Clinical Cancer Research, 25(1): 99-109 (2019), which is incorporated herein in its entirety). The one or more immunostimulatory/regulatory molecules can be administered in the form of a vector (e.g., a recombinant viral vector, such as a poxvirus or adeno associated viral vector) comprising a nucleic acid encoding one or more immunostimulatory/regulatory molecules. For example, the one or more immunostimulatory/regulatory molecules (e.g., IL-12) can be administered in the form of a DNA plasmid. Alternatively, the one or more immunostimulatory/regulatory molecules can be administered as a protein (e.g., recombinant protein).

Furthermore, such immunostimulatory/regulatory molecules and/or adjuvants can be administered to a patient in connection with an immunization regimen (such as discussed below) either as a component of the same pharmaceutical composition comprising the inventive immunogen, nucleic acid, vector, or cell, or can be administered separately, as desired.

In an embodiment, the invention further provides a method for vaccinating a human patient against HIV employing the inventive HIV-1 Env-derived immunogen and related reagents (cells comprising such, nucleic acids encoding such, vectors comprising such nucleic acids, pharmaceutical compositions). Furthermore, the invention also provides for the use of such reagents in a method of vaccinating a human patient against HIV (i.e., for use as an immunogenic reagent to vaccinate a human patient against HIV).

In accordance with the inventive method (and inventive use), a pharmaceutical composition as described herein (e.g., comprising (a) an HIV-1 Env-derived immunogen of the present invention, including a cell comprising such, for example involving embodiments in which the immunogen is membrane-bound, (b) a nucleic acid molecule encoding an inventive HIV-1 Env-derived immunogen, as described herein, (c) a genetic vector comprising such a nucleic acid, alone or in combination (as “active compound(s)”) is administered to a human patient under conditions sufficient for the patient to mount an immune response against HIV.

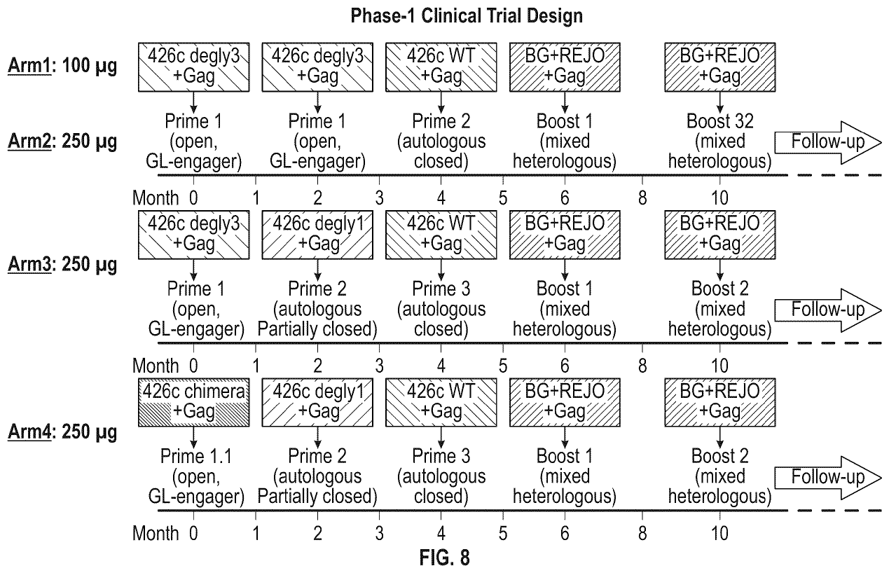

The route and location of administration can be selected, as appropriate, by the attending physician or practitioner to achieve vaccination of the patient. Thus, for example, the vaccination can be achieved by injection, aerosol, topical administration, or otherwise, depending on the manner by which the composition employed to vaccinate the patient is formulated. Moreover, the location of administration similarly can be selected by the attending physician or practitioner, and can include, for example, intramuscular or intravenous injection into the arms, legs, or other location, or the inhalation or nasal administration of an aerosol, or by the oral route or by topical application to the genital mucosa, among other routes of administration. Moreover, the dosage administered to the patient also can be selected by the attending physician or practitioner. A suitable dosage of a protein/polypeptide HIV-1 Env-derived immunogen of the present invention can be, for example, about 100 μg or about 250 μg (see, e.g., FIG. 8), but any other suitable dosage of the inventive immunogen can appropriately be selected by the attending physician or practitioner.

It will be observed that the inventive immunogen is derived from the Envelope (Env) protein of HIV-1, as described herein. Thus, typically, the inventive method and use is employed to vaccinate the human patient against HIV-1. However, the method and use can be employed to vaccinate the human patient against other HIVs, such as HIV-2.

While any suitable regimen for administering the inventive proteinaceous Env-derived immunogen, cell, nucleic acid, vector, and/or composition can be employed in the context of the present invention to vaccinate the human patient against HIV, desirably the regimen includes a prime/boost vaccination regimen. Such regimens involve at least one prime vaccination followed by at least one boost vaccination, and often several boost vaccinations over a period of time (e.g., months, or even years). For example, the vaccination regimen can comprise a prime vaccination followed by boost vaccinations separated by intervals of from a few weeks to several months, such as from 1-10 months.

One regimen, as depicted in FIG. 8, employs an initial prime vaccination comprising 250 μg of an inventive chimeric Env-derived immunogen (“426c chimera” as noted in Arm 4) followed by two different prime vaccinations at two-month increments (up to six months post-initial prime vaccination) and followed by mixed heterogenous boosts at 6 and again at 10 months following the initial prime vaccination.

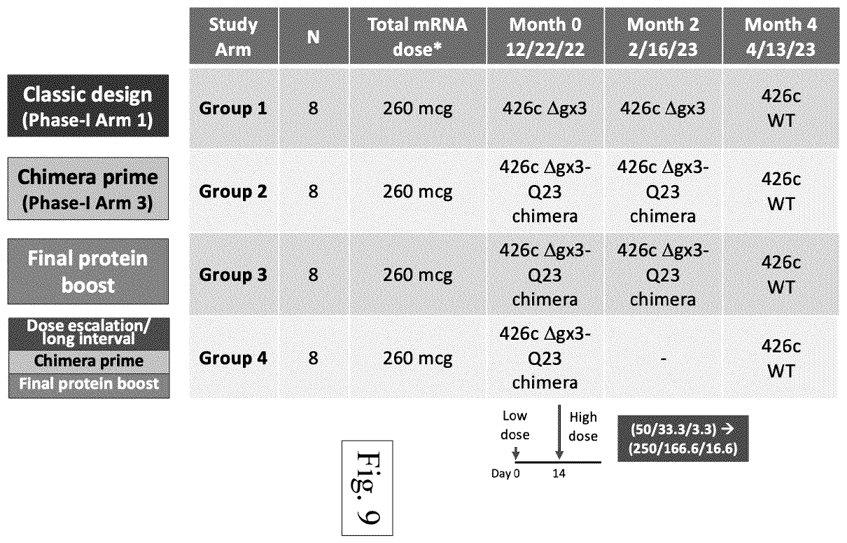

Another regimen, as depicted in FIG. 9, Arm 4, employs two initial prime inoculations of an inventive chimeric Env-derived immunogen (such as “426c Δgx3-Q23 chimera”, also referred to herein as “426c_276D_460D463D_Q23_V123” (SEQ ID NO:3)) within the first month (at day 0 and 14 in FIG. 9), with the second at an elevated dose. This can be, but need not be, followed by an extended (“long”) interval of at least four months until the next (boost) dose.

Preferably, when the inventive proteinaceous Env-derived immunogen, cell, nucleic acid, vector, and/or composition is employed in the context of a prime/boost vaccination regimen, the inventive reagent constitutes the initial prime vaccination (i.e., the first vaccine administration beginning the entire vaccination regimen). This enables the inventive immunogen to most effectively prime precursor B cells, i.e., activating and expanding naïve B cells expressing unmutated germline antibodies of various bNAb lineages. This facilitates the inventive HIV-1 Env-derived immunogen inducing multiple germline bNABs from the immunization.

Following the prime vaccination(s), in accordance with the inventive method and use, the inventive reagent (i.e., the (a) HIV-1 Env-derived immunogen of the present invention, including a cell comprising such, for example involving embodiments in which the immunogen is membrane-bound, (b) nucleic acid molecule encoding an inventive HIV-1 Env-derived immunogen, as described herein, (c) genetic vector comprising such a nucleic acid, alone or in combination thereafter, (d) composition comprising any or a combination of these) can also comprise one or more of the boost vaccinations, although, for immunization against HIV, boost vaccinations preferably employ immunogens different than the prime vaccination. Indeed, boosting with heterologous Envs, which differ from the prime vaccination(s) (and also which can differ from each successive boost vaccination) is preferred. This is represented, for example, as Arm 4 in the vaccination regime diagrammed in FIG. 8. Thus, while non-limiting, one exemplary regimen for employing the inventive method and use involves an initial prime vaccination employing the inventive pharmaceutical composition, two additional prime vaccinations separated by two-month intervals at two and four months following the initial prime vaccination, and boost vaccinations at six and ten months following the initial prime vaccination. In another embodiment, as depicted in FIG. 9 (Arm 4), the first boost dose can be administered at least four weeks following the prime dose. As depicted in FIG. 8 (Arm 4), the second and third prime vaccination can comprise HIV-1 Envs, which are single-germline engagers (such as 426c decly1 and wild-type 426; see also Table 1). Also, the boost vaccinations can comprise mixed, heterogenous agents.

The following examples further illustrate the invention but, of course, should not be construed as in any way limiting its scope.

### Example 1

This example demonstrates the synthesis and successful testing of three proteinaceous HIV-1 Env-derived immunogens according to the present invention, which simultaneously engage more than one germline lineage of broadly neutralizing antibodies (bNAbs), wherein each of the bNABs exhibits specificity for a distinct neutralization site of the HIV-1 envelope.

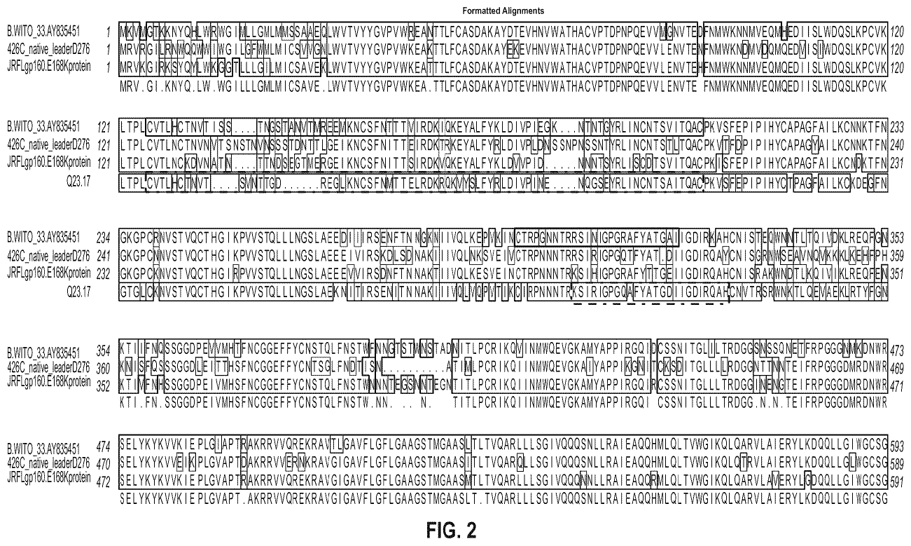

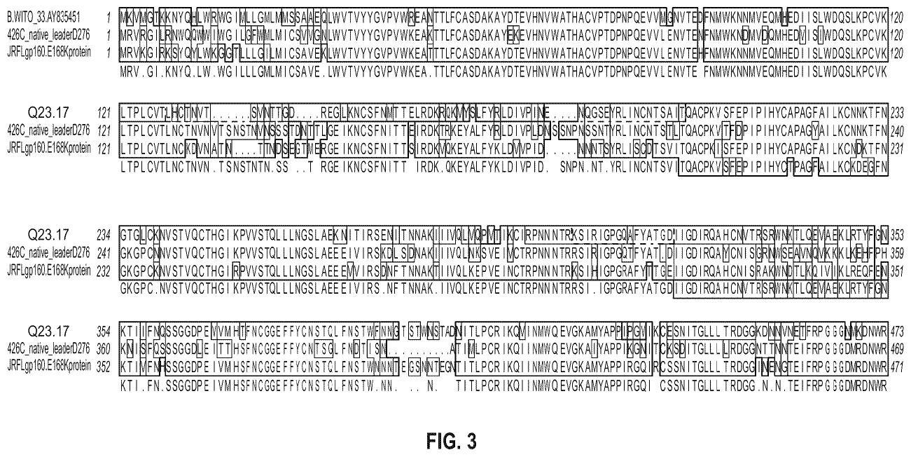

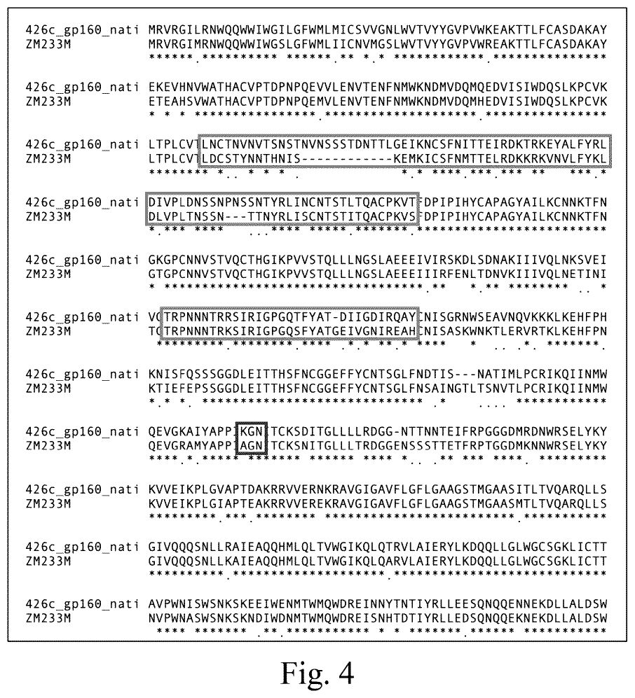

The three immunogens are each chimeric Envs, referred to herein as 426c-WITO (SEQ ID NO:1), 426-Q23.17 (SEQ ID NO:3), and 426c-ZM233 (SEQ ID NO:5). These were derived by the replacement of the V1-V2-V3 loop region at the trimer apex of 426c-deglyco3 (a potent engager of VRC01 and related germline precursor antibodies) with the homologous regions of either WITO or Q23.17 or ZM233 (three potent engagers of CH01 and related germline precursor antibodies). This achievement was aided by a structure-guided rational design of different chimeric constructs to rationally harmonize the V1-V2-V3 loop sequence grafts with the receiving core 426c-deglyco3 molecule to avoid potential molecular clashes. Sequence comparisons, such as those presented in FIGS. 2, 3, and 4, aided in this rational design effort. Individual residues were visualized within the context of the folded structure of the Env trimer, and the amino acid substitutions occurring in the engrafted sequence were projected in order to ascertain potential clashes or interference with the correct folding of the protein. When potential clashes or interference were predicted, the amino acid was replaced with the original residue present in the receiving Env sequence (426c).

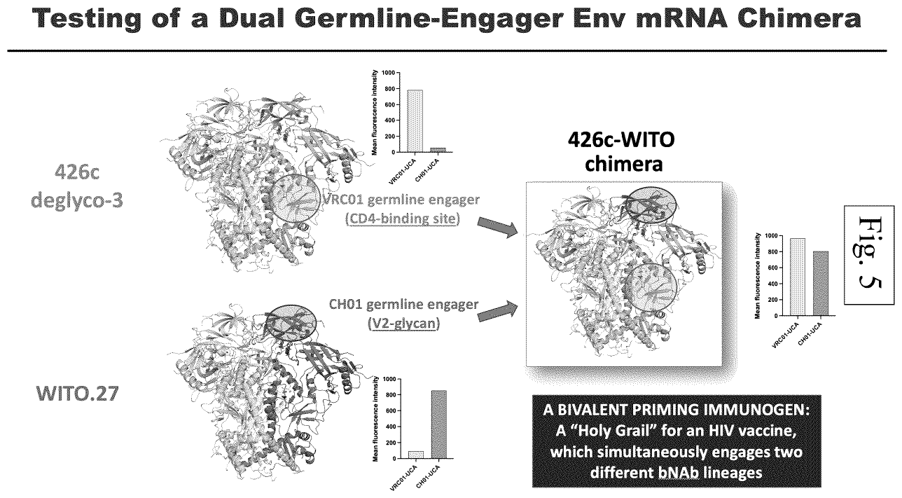

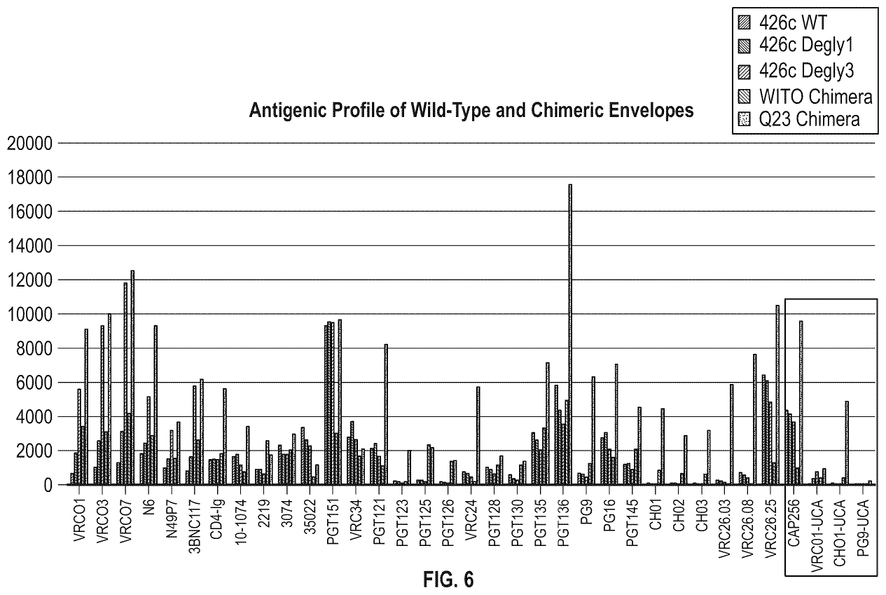

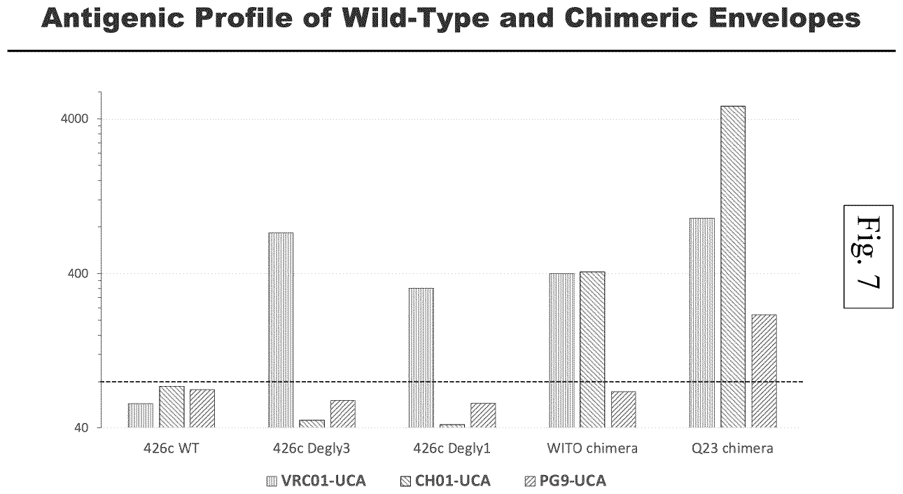

For experimental investigation, each of these three molecules was synthesized and expressed in mammalian cells (293T cells). The cells were processed and then subjected to analysis by flow cytometry. The results demonstrated that each of the three chimeric molecules retained native-like folding (they trimerized) and also retained native-like antigenic profile when compared with the source Envs from which the respective core (426c-deglyco3) and V1-V2-V3 loop regions (WITO, WQ23.17, and ZM233, respectively) were derived. Moreover, the flow cytometry assessment revealed that, unlike the source Envs, the chimeras each were able to engage two distinct lineages of germline bNAbs directed against two independent sites of HIV-1 vulnerability (the V2 glycan and the CD4-binding site). Exemplary data are presented in FIGS. 5, 6, and 7.

### Example 2

This example describes variants derived from the three chimeric HIV-1 immunogens discussed in Example 1.

One variant derivative is 426c_276D_460D463D_WITO_V12V3_TL (SEQ ID NO: 2), which is derived from 426c_276D_460D463D_WITO_V12V3 (SEQ ID NO:1). Specifically, SEQ ID NO:2 reflects amino acid replacements that were introduced to optimize the interface between the graft and the host molecule. TL corresponds to positions 200 and 201 of 426c (originally VI in WITO, which might interfere with the best-fit domain-domain interface).

Another variant derivative is 426c_276D_460D463D_Q23_V123_PGV (SEQ ID NO: 4), which is derived from 426c_276D_460D463D_Q23_V123 (SEQ ID NO:3). Specifically, SEQ ID NO:4 reflects amino acid replacements that were introduced to optimize the interface between the graft and the host molecule. PGV corresponds to positions 440-442 of Q23.17 (originally KGN in 426c but changed to PGV to avoid interference with the best-fit domain-domain interface).

Another variant derivative is 426c_276D_460D463D_ZM233_V123_MA (SEQ ID NO: 6), which is derived from 426c_276D_460D463D_ZM233_V123 (SEQ ID NO:5). Specifically, SEQ ID NO:6 reflects amino acid replacements that were introduced to optimize the interface between the graft and the host molecule. MA corresponds to positions 328 and 329 of ZM233 (originally QA in 426c), to avoid interference with the best-fit domain-domain interface).

Additional variants include SEQ ID NOs: 9-14 and 17-22, which are SOSIP and IP modifications of SEQ ID NOs: 1-6, respectively, reflecting soluble versions of these immunogens.

### Example 3

Building on the approach outlined in Example 1, additional chimeras have been designed to achieve triple germline engagement. One such molecule was designed by modifying the sequence of 426c-ZM233 (discussed above) to also permit it to engage the V3 glycan supersite of the HIV-1 envelope protein. The sequence of this chimera, termed “426c ZM_RC1_triple,” is presented herein as SEQ ID NO:7.

Another such molecule was similarly designed by modifying the sequence of 426c_276D_460D463D_Q23_V123 (discussed above) to also permit it to engage the V3 glycan supersite. The sequence of this chimera, termed “426c_Q23_RC1_triple,” is presented herein as SEQ ID NO:8.

Additional variants include SEQ ID NOs: 15, 16, 23, and 24, which are SOSIP and IP modifications of SEQ ID NOs: 7 and 8, respectively, reflecting soluble versions of these triple germline-engaging immunogens.

### Example 4

This example evinces the successful use of a chimera according to the invention as a prime immunogen in vivo, using Macaques as an experimental model.

Four cohorts of eight Macaques were immunized in accordance with the scheme presented in FIG. 9. For Arm 1, 426c Δgx3 was employed in a classic prime (cp) regimen. For Arms 2 and 3, the 426c Δgx3-Q23 chimera (also referred to herein as “426c 276D_460D463D_Q23_V123” (SEQ ID NO:3)) was employed in a cp regimen. For Arm 4, 426c Δgx3-Q23 chimera was employed in a dose-escalation/long-interval prime regimen according to which, during month 0, the dosage was repeated and escalated as indicated with no dosing at month 2. Not shown in FIG. 9 are periods after Month 4. The study plan includes booster doses of all cohorts of Macaques at Months 6 and 9 with BG505 and REJO. At Month 12, Arms 1 and 2 are to receive another booster dose of BG505 and REJO RNA, while Arms 3 and 4 are to receive BG505 and REJO protein.

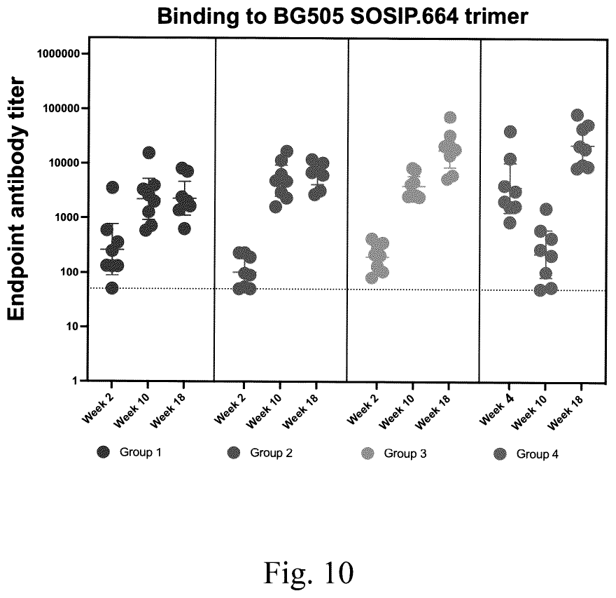

Blood was drawn from each Macaque in the study at weeks 2, 10, and 18. The samples were assayed for the antibody binding titers to a heterologous strain trimer (BG505 SOSIP.664 trimer). The results are presented in FIG. 10, and they reveal that prime dosing with the 426-Q23 chimera results in a higher immunogenicity than when the 426 wild-type strain in all study arms.

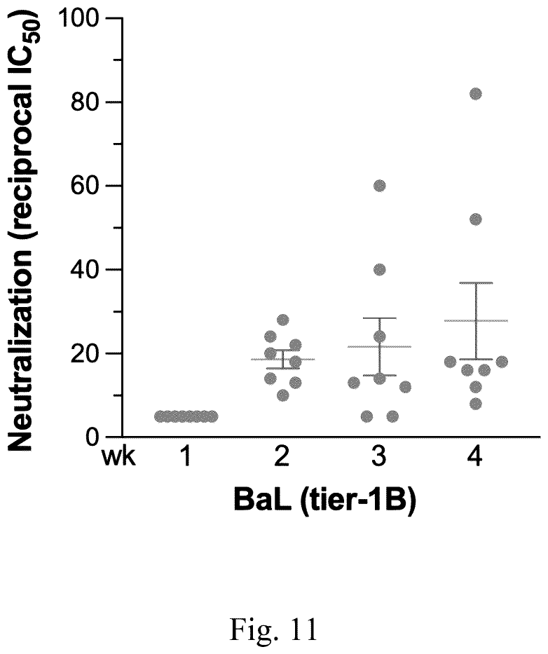

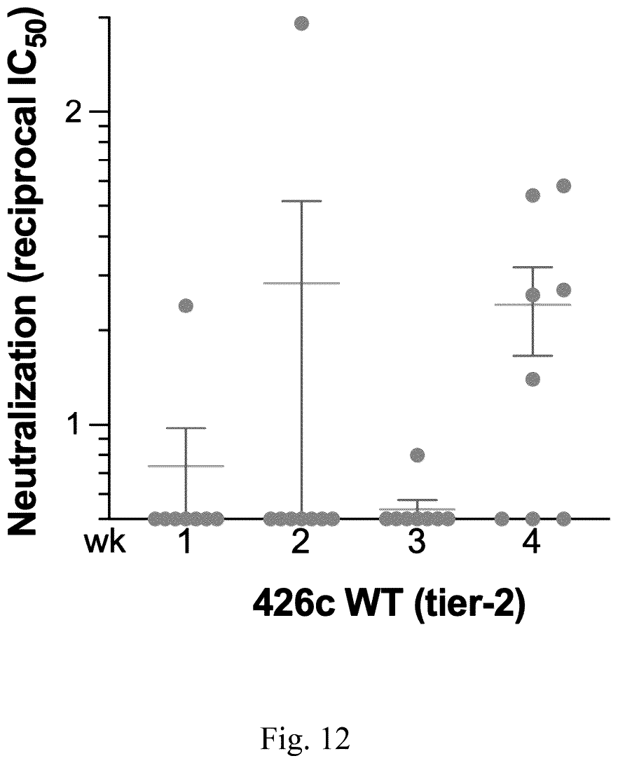

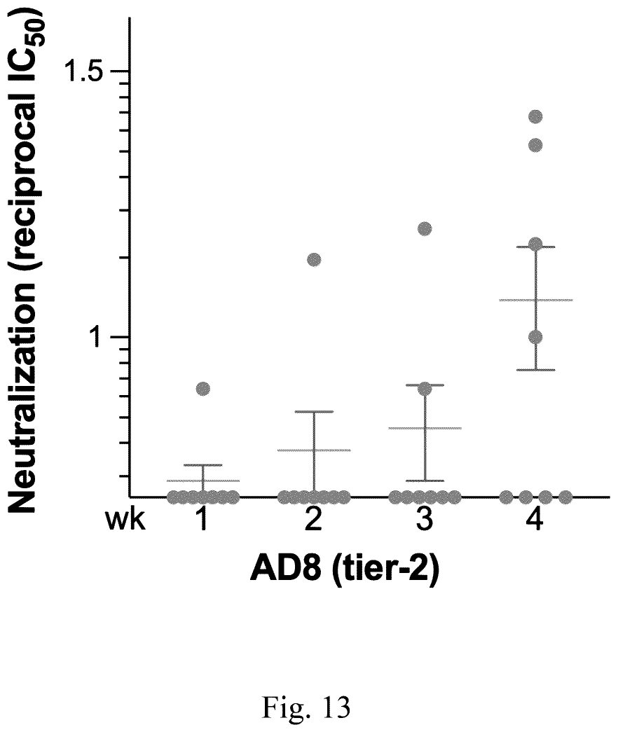

Using samples drawn from the Macaques at 18 weeks, neutralization assays were performed using three heterologous strains (not the 426c Δgx3-Q23 chimera used for immunization): BaL (a Tier-1B HIV-1 strain), 426 wild-type (WT) (a Tier-2 HIV-1 strain), and AD8 (also a Tier-2 HIV-1 strain). The data from these experiments are presented in FIGS. 11-13. These data reveal that 426c Δgx3-Q23 chimera induces neutralizing antibodies as well as or better than wild-type in all 3 study arms. Moreover, Macaques in Arm 4 were observed to develop neutralization, even against Tier-2 strains, which are very difficult to neutralize, after only three immunizations (i.e., the initial dose-escalation prime in Month 0 and one boost dose at Month 4). This result for the Macaques in Arm 4 was particularly surprising.

To summarize, the results of the in vivo experiments reported in this Example reveal that an inventive chimera (426c Δgx3-Q23) is a highly effective immunogen against multiple strains of HIV-1 when used as a prime immunogen according to different protocols.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

SEQUENCES

SEQ ID NO: 1- >426c_276D_460D463D_WITO_V12V3 (a membrane-bound ENV gp150
(1-745))
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLHCTNVTISSTNGSTANVTMREEMKNCSFNTTTVIRDKIQKEYAL
FYKLDIVPIEGKNTNTGYRLINCNTSVITQACPKVTFDPIPIHYCAPAGYAILKCNNKTFN
GKGPCNNVSTVQCTHGIKPVVSTQLLLNGSLAEEEIVIRSKDLSDNAKIIIVQLNKSVEIV
CTRPGNNTRRSINIGPGRAFYATGAIIGDIRQAYCNISGRNWSEAVNQVKKKLKEHFPHK
NISFQSSSGGDLEITTHSFNCGGEFFYCNTSGLENDTISNATIMLPCRIKQIINMWQEVGK
AIYAPPIKGNITCKSDITGLLLLRDGGDTTDNTEIFRPGGGDMRDNWRSELYKYKVVEIK
PLGVAPTDAKRRVVERNKRAVGIGAVFLGFLGAAGSTMGAASITLTVQARQLLSGIVQQ
QSNLLRAIEAQQHMLQLTVWGIKQLQTRVLAIERYLKDQQLLGLWGCSGKLICTTAVP
WNISWSNKSKEEIWENMTWMQWDREINNYTNTIYRLLEESQNQQENNEKDLLALDSW
NNLWNWFNITNWLWYIKIFIMIVGGLIGLRIIFIVLSIVNRVRQGYSPLSFQTLIPNPRGPD
RPGGIEEEGGEQDRDRS
SEQ ID NO: 2- >426c_ 276D_460D463D_WITO_V12V3_TL (a membrane-bound ENV
gp150 (1-745))
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLHCTNVTISSTNGSTANVTMREEMKNCSFNTTTVIRDKIQKEYAL
FYKLDIVPIEGKNTNTGYRLINCNTSVTLQACPKVTFDPIPIHYCAPAGYAILKCNNKTFN
GKGPCNNVSTVQCTHGIKPVVSTQLLLNGSLAEEEIVIRSKDLSDNAKIIIVQLNKSVEIV
CTRPGNNTRRSINIGPGRAFYATGAIIGDIRQAYCNISGRNWSEAVNQVKKKLKEHFPHK
NISFQSSSGGDLEITTHSFNCGGEFFYCNTSGLFNDTISNATIMLPCRIKQIINMWQEVGK
AIYAPPIKGNITCKSDITGLLLLRDGGDTTDNTEIFRPGGGDMRDNWRSELYKYKVVEIK
PLGVAPTDAKRRVVERNKRAVGIGAVFLGFLGAAGSTMGAASITLTVQARQLLSGIVQQ
QSNLLRAIEAQQHMLQLTVWGIKQLQTRVLAIERYLKDQQLLGLWGCSGKLICTTAVP
WNISWSNKSKEEIWENMTWMQWDREINNYTNTIYRLLEESQNQQENNEKDLLALDSW
NNLWNWFNITNWLWYIKIFIMIVGGLIGLRIIFIVLSIVNRVRQGYSPLSFQTLIPNPRGPD
RPGGIEEEGGEQDRDRS
SEQ ID NO: 3- >426c_276D_460D463D_Q23_V123 (a membrane-bound ENV gp150
(1-745))
MR VRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLHCTNVTSVNTTGDREGLKNCSFNMTTELRDKRQKVYSLFYRL
DIVPINENQGSEYRLINCNTSAITQACPKVTFDPIPIHYCAPAGYAILKCNNKTFNGKGPC
NNVSTVQCTHGIKPVVSTQLLLNGSLAEEEIVIRSKDLSDNAKIIIVQLNKSVEIVCTRPNN
NTRKSIRIGPGQAFYATGDIIGDIRQAYCNISGRNWSEAVNQVKKKLKEHFPHKNISFQSS
SGGDLEITTHSFNCGGEFFYCNTSGLFNDTISNATIMLPCRIKQIINMWQEVGKAIYAPPIK
GNITCKSDITGLLLLRDGGDTTDNTEIFRPGGGDMRDNWRSELYKYKVVEIKPLGVAPT
DAKRRVVERNKRAVGIGAVFLGFLGAAGSTMGAASITLTVQARQLLSGIVQQQSNLLR
AIEAQQHMLQLTVWGIKQLQTRVLAIERYLKDQQLLGLWGCSGKLICTTAVPWNISWS
NKSKEEIWENMTWMQWDREINNYTNTIYRLLEESQNQQENNEKDLLALDSWNNLWN
WFNITNWLWYIKIFIMIVGGLIGLRIIFIVLSIVNRVRQGYSPLSFQTLIPNPRGPDRPGGIEE
EGGEQDRDRS
SEQ ID NO: 4- >426c_276D_460D463D_Q23_V123_PGV (a membrane-bound ENV gp150
(1-745))
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLHCTNVTSVNTTGDREGLKNCSFNMTTELRDKRQKVYSLFYRL
DIVPINENQGSEYRLINCNTSAITQACPKVTFDPIPIHYCAPAGYAILKCNNKTFNGKGPC
NNVSTVQCTHGIKPVVSTQLLLNGSLAEEEIVIRSKDLSDNAKIIIVQLNKSVEIVCTRPNN
NTRKSIRIGPGQAFYATGDIIGDIRQAYCNISGRNWSEAVNQVKKKLKEHFPHKNISFQSS
SGGDLEITTHSFNCGGEFFYCNTSGLFNDTISNATIMLPCRIKQIINMWQEVGKAIYAPPIP
GVITCKSDITGLLLLRDGGDTTDNTEIFRPGGGDMRDNWRSELYKYKVVEIKPLGVAPT
DAKRRVVERNKRAVGIGAVFLGFLGAAGSTMGAASITLTVQARQLLSGIVQQQSNLLR
AIEAQQHMLQLTVWGIKQLQTRVLAIERYLKDQQLLGLWGCSGKLICTTAVPWNISWS
NKSKEEIWENMTWMQWDREINNYTNTIYRLLEESQNQQENNEKDLLALDSWNNLWN
WFNITNWLWYIKIFIMIVGGLIGLRIIFIVLSIVNRVRQGYSPLSFQTLIPNPRGPDRPGGIEE
EGGEQDRDRS
SEQ ID NO: 5- >426c_276D_460D463D_ZM233_V123 (a membrane-bound ENV gp150
(1-745))
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCV TLDCSTYNNTHNISKEMKICSFNMTTELRDKKRKVNVLFYKLDLV
PLTNSSNTTNYRLISCNTSTITQACPKVTFDPIPIHYCAPAGYAILKCNNKTFNGKGPCNN
VSTVQCTHGIKPVVSTQLLLNGSLAEEEIVIRSKDLSDNAKIIIVQLNKSVEIVC TRPNNN
TRKSIRIGPGQSFYATGEIVGNIREAHCNISGRNWSEAVNQVKKKLKEHFPHKNISFQSSS
GGDLEITTHSFNCGGEFFYCNTSGLFNDTISNATIMLPCRIKQIINMWQEVGKAIYAPPIK
GNITCKSDITGLLLLRDGGDTTDNTEIFRPGGGDMRDNWRSELYKYKVVEIKPLGVAPT
DAKRRVVERNKRAVGIGAVFLGFLGAAGSTMGAASITLTVQARQLLSGIVQQQSNLLR
AIEAQQHMLQLTVWGIKQLQTRVLAIERYLKDQQLLGLWGCSGKLICTTAVPWNISWS
NKSKEEIWENMTWMQWDREINNYTNTIYRLLEESQNQQENNEKDLLALDSWNNLWN
WFNITNWLWYIKIFIMIVGGLIGLRIIFIVLSIVNRVRQGYSPLSFQTLIPNPRGPDRPGGIEE
EGGEQDRDRS
SEQ ID NO: 6- >426c_276D_460D463D_ZM233_V123_MA (a membrane-bound ENV
gp150 (1-745))
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLDCSTYNNTHNISKEMKICSFNMTTELRDKKRKVNVLFYKLDLV
PLTNSSNTTNYRLISCNTSTITQACPKVTFDPIPIHYCAPAGYAILKCNNKTFNGKGPCNN
VSTVQCTHGIKPVVSTQLLLNGSLAEEEIVIRSKDLSDNAKIIIVQLNKSVEIVCTRPNNN
TRKSIRIGPGQSFYATGEIVGNIREAHCNISGRNWSEAVNQVKKKLKEHFPHKNISFQSSS
GGDLEITTHSFNCGGEFFYCNTSGLFNDTISNATIMLPCRIKQIINMWQEVGKAMYAPPIA
GNITCKSDITGLLLLRDGGDTTDNTEIFRPGGGDMRDNWRSELYKYKVVEIKPLGVAPT
DAKRRVVERNKRAVGIGAVFLGFLGAAGSTMGAASITLTVQARQLLSGIVQQQSNLLR
AIEAQQHMLQLTVWGIKQLQTRVLAIERYLKDQQLLGLWGCSGKLICTTAVPWNISWS
NKSKEEIWENMTWMQWDREINNYTNTIYRLLEESQNQQENNEKDLLALDSWNNLWN
WFNITNWLWYIKIFIMIVGGLIGLRIIFIVLSIVNRVRQGYSPLSFQTLIPNPRGPDRPGGIEE
EGGEQDRDRS
SEQ ID NO: 7- >426c_ZM_RC1_triple (a membrane-bound ENV gp150 (1-745))
MR VRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCV TLQCTNYAPNLLSNMRGELKQCSFNMTTELRDKKRKVNVLFYKL
DLVPLTNSSNTTNYRLISCNTSTITQACPKVTFDPIPIHYCAPAGYAILKCNNKTFNGKGP
CNNVSTVQCTHGIKPVVSTQLLLNGSLAEEEIVIRSKDLSDNAKIIIVQLNTPVQINCTRPN
NNTRKSIRIGPGQSFYATGEIIGDIRMAHCNVSKATWNEAVNQVKKKLKEHFPHKNISFQ
SSSGGDLEITTHSFNCGGEFFYCNTSGLFNDTISNATIVLPCRIKQIINMWQEVGKAMYAP
PIQGVIRCVSNITGLLLLRDGGDTTDNTEIFRPGGGDMRDNWRSELYKYKVVEIKPLGVA
PTDAKRRVVERNKRAVGIGAVFLGFLGAAGSTMGAASITLTVQARQLLSGIVQQQSNLL
RAIEAQQHMLQLTVWGIKQLQTRVLAIERYLKDQQLLGLWGCSGKLICTTAVPWNISW
SNKSKEEIWENMTWMQWDREINNYTNTIYRLLEESQNQQENNEKDLLALDSWNNLWN
WFNITNWLWYIKIFIMIVGGLIGLRIIFIVLSIVNRVRQGYSPLSFQTLIPNPRGPDRPGGIEE
EGGEQDRDRS
SEQ ID NO: 8- >426c_Q23_RC1_triple (a membrane-bound ENV gp150 (1-745))
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLHCTNVTPNLNTTGDREGLKNCSFNMTTELRDKRQKVYSLFYRL
DIVPINENQGSEYRLINCNTSAITQACPKVTFDPIPIHYCAPAGYAILKCNNKTFNGKGPC
NNVSTVQCTHGIKPVVSTQLLLNGSLAEEEIVIRSKDLSDNAKIIIVQLNKSVQINCTRPN
NNTVKSIRIGPGQAFYYFGDIIGDIRMAYCNISGRNWSEAVNQVKKKLKEHFPHKNISFQ
SSSGGDLEITTHSFNCGGEFFYCNTSGLFNDTISNDSIMLPCRIKQIINMWQEVGKAIYAPP
IPGVITCKSDITGLLLLRDGGDTTDNTEIFRPGGGDMRDNWRSELYKYKVVEIKPLGVAP
TDAKRRVVERNKRAVGIGAVFLGFLGAAGSTMGAASITLTVQARQLLSGIVQQQSNLLR
AIEAQQHMLQLTVWGIKQLQTRVLAIERYLKDQQLLGLWGCSGKLICTTAVPWNISWS
NKSKEEIWENMTWMQWDREINNYTNTIYRLLEESQNQQENNEKDLLALDSWNNLWN
WFNITNWLWYIKIFIMIVGGLIGLRIIFIVLSIVNRVRQGYSPLSFQTLIPNPRGPDRPGGIEE
EGGEQDRDRS
SEQ ID NO: 9- 426c_276D_460D463D_WITO_V12V3.SOSIP (a SOSIP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLHCTNVTISSTNGSTANVTMREEMKNCSFNTTTVIRDKIQKEYAL
FYKLDIVPIEGKNTNTGYRLINCNTSVITQACPKVTFDPIPIHYCAPAGYAILKCNNKTFN
GKGPCNNVSTVQCTHGIKPVVSTQLLLNGSLAEEEIVIRSKDLSDNAKIIIVQLNKSVEIV
CTRPGNNTRRSINIGPGRAFYATGAIIGDIRQAYCNISGRNWSEAVNQVKKKLKEHFPHK
NISFQSSSGGDLEITTHSFNCGGEFFYCNTSGLENDTISNATIMLPCRIKQIINMWQEVGK
AIYAPPIKGNITCKSDITGLLLLRDGGDTTDNTEIFRPGGGDMRDNWRSELYKYKVVEIK
PLGVAPTDCKRRVVERRRRRRAVGIGAVFLGFLGAAGSTMGAASITLTVQARQLLSGIV
QQQSNLLRAPEAQQHMLQLTVWGIKQLQTRVLAIERYLKDQQLLGLWGCSGKLICCTA
VPWNISWSNKSKEEIWENMTWMQWDREINNYTNTIYRLLEESQNQQENNEKDLLALD
SEQ ID NO: 10- >426c_276D_460D463D_WITO_V12V3_TL.SOSIP (a SOSIP trimer)
MR VRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLHCTNVTISSTNGSTANVTMREEMKNCSFNTTTVIRDKIQKEYAL
FYKLDIVPIEGKNTNTGYRLINCNTSVTLQACPKVTFDPIPIHYCAPAGYAILKCNNKTFN
GKGPCNNVSTVQCTHGIKPVVSTQLLLNGSLAEEEIVIRSKDLSDNAKIIIVQLNKSVEIV
CTRPGNNTRRSINIGPGRAFYATGAIIGDIRQAYCNISGRNWSEAVNQVKKKLKEHFPHK
NISFQSSSGGDLEITTHSFNCGGEFFYCNTSGLFNDTISNATIMLPCRIKQIINMWQEVGK
AIYAPPIKGNITCKSDITGLLLLRDGGDTTDNTEIFRPGGGDMRDNWRSELYKYKVVEIK
PLGVAPTDCKRRVVERRRRRRAVGIGAVFLGFLGAAGSTMGAASITLTVQARQLLSGIV
QQQSNLLRAPEAQQHMLQLTVWGIKQLQTRVLAIERYLKDQQLLGLWGCSGKLICCTA
VPWNISWSNKSKEEIWENMTWMQWDREINNYTNTIYRLLEESQNQQENNEKDLLALD
SEQ ID NO: 11- >426c_276D_460D463D_Q23_V123.SOSIP (a SOSIP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLHCTNVTSVNTTGDREGLKNCSFNMTTELRDKRQKVYSLFYRL
DIVPINENQGSEYRLINCNTSAITQACPKVTFDPIPIHYCAPAGYAILKCNNKTFNGKGPC
NNVSTVQCTHGIKPVVSTQLLLNGSLAEEEIVIRSKDLSDNAKIIIVQLNKSVEIVCTRPNN
NTRKSIRIGPGQAFYATGDIIGDIRQAYCNISGRNWSEAVNQVKKKLKEHFPHKNISFQSS
SGGDLEITTHSFNCGGEFFYCNTSGLFNDTISNATIMLPCRIKQIINMWQEVGKAIYAPPIK
GNITCKSDITGLLLLRDGGDTTDNTEIFRPGGGDMRDNWRSELYKYKVVEIKPLGVAPT
DCKRRVVERRRRRRAVGIGAVFLGFLGAAGSTMGAASITLTVQARQLLSGIVQQQSNLL
RAPEAQQHMLQLTVWGIKQLQTRVLAIERYLKDQQLLGLWGCSGKLICCTAVPWNISW
SNKSKEEIWENMTWMQWDREINNYTNTIYRLLEESQNQQENNEKDLLALD
SEQ ID NO: 12 ->426c_276D_460D463D_Q23_V123_PGV.SOSIP (a SOSIP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLHCTNVTSVNTTGDREGLKNCSFNMTTELRDKRQKVYSLFYRL
DIVPINENQGSEYRLINCNTSAITQACPKVTFDPIPIHYCAPAGYAILKCNNKTFNGKGPC
NNVSTVQCTHGIKPVVSTQLLLNGSLAEEEIVIRSKDLSDNAKIIIVQLNKSVEIVCTRPNN
NTRKSIRIGPGQAFYATGDIIGDIRQAYCNISGRNWSEAVNQVKKKLKEHFPHKNISFQSS
SGGDLEITTHSFNCGGEFFYCNTSGLFNDTISNATIMLPCRIKQIINMWQEVGKAIYAPPIP
GVITCKSDITGLLLLRDGGDTTDNTEIFRPGGGDMRDNWRSELYKYKVVEIKPLGVAPT
DCKRRVVERRRRRRAVGIGAVFLGFLGAAGSTMGAASITLTVQARQLLSGIVQQQSNLL
RAPEAQQHMLQLTVWGIKQLQTRVLAIERYLKDQQLLGLWGCSGKLICCTAVPWNISW
SNKSKEEIWENMTWMQWDREINNYTNTIYRLLEESQNQQENNEKDLLALD
SEQ ID NO: 13- >426c_276D_460D463D_ZM233_V123.SOSIP (a SOSIP trimer)
MR VRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCV TLDCSTYNNTHNISKEMKICSFNMTTELRDKKRKVNVLFYKLDLV
PLTNSSNTTNYRLISCNTSTITQACPKVTFDPIPIHYCAPAGYAILKCNNKTFNGKGPCNN
VSTVQCTHGIKPVVSTQLLLNGSLAEEEIVIRSKDLSDNAKIIIVQLNKSVEIVC TRPNNN
TRKSIRIGPGQSFYATGEIVGNIREAHCNISGRNWSEAVNQVKKKLKEHFPHKNISFQSSS
GGDLEITTHSFNCGGEFFYCNTSGLFNDTISNATIMLPCRIKQIINMWQEVGKAIYAPPIK
GNITCKSDITGLLLLRDGGDTTDNTEIFRPGGGDMRDNWRSELYKYKVVEIKPLGVAPT
DCKRRVVERRRRRRAVGIGAVELGFLGAAGSTMGAASITLTVQARQLLSGIVQQQSNLL
RAPEAQQHMLQLTVWGIKQLQTRVLAIERYLKDQQLLGLWGCSGKLICCTAVPWNISW
SNKSKEEIWENMTWMQWDREINNYTNTIYRLLEESQNQQENNEKDLLALD
SEQ ID NO: 14- >426c_276D_460D463D_ZM233_V123_MA.SOSIP (a SOSIP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCV TLDCSTYNNTHNISKEMKICSFNMTTELRDKKRKVNVLFYKLDLV
PLTNSSNTTNYRLISCNTSTITQACPKVTFDPIPIHYCAPAGYAILKCNNKTFNGKGPCNN
VSTVQCTHGIKPVVSTQLLLNGSLAEEEIVIRSKDLSDNAKIIIVQLNKSVEIVC TRPNNN
TRKSIRIGPGQSFYATGEIVGNIREAHCNISGRNWSEAVNQVKKKLKEHFPHKNISFQSSS
GGDLEITTHSFNCGGEFFYCNTSGLENDTISNATIMLPCRIKQIINMWQEVGKAMYAPPIA
GNITCKSDITGLLLLRDGGDTTDNTEIFRPGGGDMRDNWRSELYKYKVVEIKPLGVAPT
DCKRRVVERRRRRRAVGIGAVFLGFLGAAGSTMGAASITLTVQARQLLSGIVQQQSNLL
RAPEAQQHMLQLTVWGIKQLQTRVLAIERYLKDQQLLGLWGCSGKLICCTAVPWNISW
SNKSKEEIWENMTWMQWDREINNYTNTIYRLLEESQNQQENNEKDLLALD
SEQ ID NO: 15- >426c_ZM_RC1_triple.SOSIP (a SOSIP trimer)
MR VRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCV TLQCTNYAPNLLSNMRGELKQCSFNMTTELRDKKRKVNVLFYKL
DLVPLTNSSNTTNYRLISCNTSTITQACPKVTFDPIPIHYCAPAGYAILKCNNKTFNGKGP
CNNVSTVQCTHGIKPVVSTQLLLNGSLAEEEIVIRSKDLSDNAKIIIVQLNTPVQINCTRPN
NNTRKSIRIGPGQSFYATGEIIGDIRMAHCNVSKATWNEAVNQVKKKLKEHFPHKNISFQ
SSSGGDLEITTHSFNCGGEFFYCNTSGLFNDTISNATIVLPCRIKQIINMWQEVGKAMYAP
PIQGVIRCVSNITGLLLLRDGGDTTDNTEIFRPGGGDMRDNWRSELYKYKVVEIKPLGVA
PTDCKRRVVERRRRRRAVGIGAVFLGFLGAAGSTMGAASITLTVQARQLLSGIVQQQSN
LLRAPEAQQHMLQLTVWGIKQLQTRVLAIERYLKDQQLLGLWGCSGKLICCTAVPWNI
SWSNKSKEEIWENMTWMQWDREINNYTNTIYRLLEESQNQQENNEKDLLALD
SEQ ID NO: 16- >426c_Q23_RC1_triple.SOSIP (a SOSIP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLHCTNVTPNLNTTGDREGLKNCSFNMTTELRDKRQKVYSLFYRL
DIVPINENQGSEYRLINCNTSAITQACPKVTFDPIPIHYCAPAGYAILKCNNKTFNGKGPC
NNVSTVQCTHGIKPVVSTQLLLNGSLAEEEIVIRSKDLSDNAKIIIVQLNKSVQINCTRPN
NNTVKSIRIGPGQAFYYFGDIIGDIRMAYCNISGRNWSEAVNQVKKKLKEHFPHKNISFQ
SSSGGDLEITTHSFNCGGEFFYCNTSGLFNDTISNDSIMLPCRIKQIINMWQEVGKAIYAPP
IPGVITCKSDITGLLLLRDGGDTTDNTEIFRPGGGDMRDNWRSELYKYKVVEIKPLGVAP
TDCKRRVVERRRRRRAVGIGAVFLGFLGAAGSTMGAASITLTVQARQLLSGIVQQQSNL
LRAPEAQQHMLQLTVWGIKQLQTRVLAIERYLKDQQLLGLWGCSGKLICCTAVPWNIS
WSNKSKEEIWENMTWMQWDREINNYTNTIYRLLEESQNQQENNEKDLLALD
SEQ ID NO: 17- >426c_276D_460D463D_WITO_V12V3.IP (an IP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWC
QSLKPCVKLTPLCVTLHCTNVTISSTNGSTANVTMREEMKNCSFNTTTVIRDKIQKEYAL
FYKLDIVPIEGKNTNTGYRLINCNTSVITQACPKVTFDPIPIHYCAPAGYAILKCNNKTFN
GKGPCNNVSTVQCTHGIKPVVSTQLLLNGSLAEEEIVIRSKDLSDNAKIIIVQLNKSVEIV
CTRPGNNTRRSINIGPGRAFYATGAIIGDIRQAYCNISGRNWSEAVNQVKKKLKEHFPHK
NISFQSSSGGDLEITTHSFNCGGEFFYCNTSGLENDTISNATIMLPCRIKQIINMWQEVGCG
AIYAPPIKGNITCKSDITGLLLLRDGGDTTDNTEIFRPGGGDMRDNWRSELYKYKVVEIK
PLGVAPTDAKRRVVERRRRRRAVGIGAVFLGFLGAAGSTMGAASITLTVQARQLLSGIV
QQQSNLLRAPEAQQHMLQLTVWGIKQLQTRVLAIERYLKDQQLLGLWGCSGKLICTTA
VPWNISWSNKSKEEIWENMTWMQWDREINNYTNTIYRLLEESQNQQENNEKDLLALD
SEQ ID NO: 18- >426c_276D_460D463D_WITO_V12V3_TL.IP (an IP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWC
QSLKPCVKLTPLCVTLHCTNVTISSTNGSTANVTMREEMKNCSFNTTTVIRDKIQKEYAL
FYKLDIVPIEGKNTNTGYRLINCNTSVTLQACPKVTFDPIPIHYCAPAGYAILKCNNKTEN
GKGPCNNVSTVQCTHGIKPVVSTQLLLNGSLAEEEIVIRSKDLSDNAKIIIVQLNKSVEIV
CTRPGNNTRRSINIGPGRAFYATGAIIGDIRQAYCNISGRNWSEAVNQVKKKLKEHFPHK
NISFQSSSGGDLEITTHSFNCGGEFFYCNTSGLFNDTISNATIMLPCRIKQIINMWQEVGCG
AIYAPPIKGNITCKSDITGLLLLRDGGDTTDNTEIFRPGGGDMRDNWRSELYKYKVVEIK
PLGVAPTDAKRRVVERRRRRRAVGIGAVFLGFLGAAGSTMGAASITLTVQARQLLSGIV
QQQSNLLRAPEAQQHMLQLTVWGIKQLQTRVLAIERYLKDQQLLGLWGCSGKLICTTA
VPWNISWSNKSKEEIWENMTWMQWDREINNYTNTIYRLLEESQNQQENNEKDLLALD
SEQ ID NO: 19- >426c_276D_460D463D_Q23_V123.IP (an IP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWC
QSLKPCVKLTPLCVTLHCTNVTSVNTTGDREGLKNCSFNMTTELRDKRQKVYSLFYRL
DIVPINENQGSEYRLINCNTSAITQACPKVTFDPIPIHYCAPAGYAILKCNNKTFNGKGPC
NNVSTVQCTHGIKPVVSTQLLLNGSLAEEEIVIRSKDLSDNAKIIIVQLNKSVEIVCTRPNN
NTRKSIRIGPGQAFYATGDIIGDIRQAYCNISGRNWSEAVNQVKKKLKEHFPHKNISFQSS
SGGDLEITTHSFNCGGEFFYCNTSGLFNDTISNATIMLPCRIKQIINMWQEVGCGAIYAPPI
KGNITCKSDITGLLLLRDGGDTTDNTEIFRPGGGDMRDNWRSELYKYKVVEIKPLGVAP
TDAKRRVVERRRRRRAVGIGAVFLGFLGAAGSTMGAASITLTVQARQLLSGIVQQQSNL
LRAPEAQQHMLQLTVWGIKQLQTRVLAIERYLKDQQLLGLWGCSGKLICTTAVPWNIS
WSNKSKEEIWENMTWMQWDREINNYTNTIYRLLEESQNQQENNEKDLLALD
SEQ ID NO: 20- >426c_276D_460D463D_Q23_V123_PGV.IP (an IP trimer)
MR VRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWC
QSLKPCVKLTPLCVTLHCTNVTSVNTTGDREGLKNCSFNMTTELRDKRQKVYSLFYRL
DIVPINENQGSEYRLINCNTSAITQACPKVTFDPIPIHYCAPAGYAILKCNNKTFNGKGPC
NNVSTVQCTHGIKPVVSTQLLLNGSLAEEEIVIRSKDLSDNAKIIIVQLNKSVEIVCTRPNN
NTRKSIRIGPGQAFYATGDIIGDIRQAYCNISGRNWSEAVNQVKKKLKEHFPHKNISFQSS
SGGDLEITTHSFNCGGEFFYCNTSGLFNDTISNATIMLPCRIKQIINMWQEVGCGAIYAPPI
PGVITCKSDITGLLLLRDGGDTTDNTEIFRPGGGDMRDNWRSELYKYKVVEIKPLGVAP
TDAKRRVVERRRRRRAVGIGAVELGFLGAAGSTMGAASITLTVQARQLLSGIVQQQSNL
LRAPEAQQHMLQLTVWGIKQLQTRVLAIERYLKDQQLLGLWGCSGKLICTTAVPWNIS
WSNKSKEEIWENMTWMQWDREINNYTNTIYRLLEESQNQQENNEKDLLALD
SEQ ID NO: 21- >426c_276D_460D463D_ZM233_V123.IP (an IP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWC
QSLKPCVKLTPLCVTLDCSTYNNTHNISKEMKICSFNMTTELRDKKRKVNVLFYKLDLV
PLTNSSNTTNYRLISCNTSTITQACPKVTFDPIPIHYCAPAGYAILKCNNKTFNGKGPCNN
VSTVQCTHGIKPVVSTQLLLNGSLAEEEIVIRSKDLSDNAKIIIVQLNKSVEIVC TRPNNN
TRKSIRIGPGQSFYATGEIVGNIREAHCNISGRNWSEAVNQVKKKLKEHFPHKNISFQSSS
GGDLEITTHSFNCGGEFFYCNTSGLFNDTISNATIMLPCRIKQIINMWQEVGCGAIYAPPI
KGNITCKSDITGLLLLRDGGDTTDNTEIFRPGGGDMRDNWRSELYKYKVVEIKPLGVAP
TDAKRRVVERRRRRRAVGIGAVFLGFLGAAGSTMGAASITLTVQARQLLSGIVQQQSNL
LRAPEAQQHMLQLTVWGIKQLQTRVLAIERYLKDQQLLGLWGCSGKLICTTAVPWNIS
WSNKSKEEIWENMTWMQWDREINNYTNTIYRLLEESQNQQENNEKDLLALD
SEQ ID NO: 22- >426c_276D_460D463D_ZM233_V123_MA.IP (an IP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWC
QSLKPCVKLTPLCV TLDCSTYNNTHNISKEMKICSFNMTTELRDKKRKVNVLFYKLDLV
PLTNSSNTTNYRLISCNTSTITQACPKVTFDPIPIHYCAPAGYAILKCNNKTFNGKGPCNN
VSTVQCTHGIKPVVSTQLLLNGSLAEEEIVIRSKDLSDNAKIIIVQLNKSVEIVC TRPNNN
TRKSIRIGPGQSFYATGEIVGNIREAHCNISGRNWSEAVNQVKKKLKEHFPHKNISFQSSS
GGDLEITTHSFNCGGEFFYCNTSGLFNDTISNATIMLPCRIKQIINMWQEVGCGAMYAPPI
AGNITCKSDITGLLLLRDGGDTTDNTEIFRPGGGDMRDNWRSELYKYKVVEIKPLGVAP
TDAKRRVVERRRRRRAVGIGAVELGFLGAAGSTMGAASITLTVQARQLLSGIVQQQSNL
LRAPEAQQHMLQLTVWGIKQLQTRVLAIERYLKDQQLLGLWGCSGKLICTTAVPWNIS
WSNKSKEEIWENMTWMQWDREINNYTNTIYRLLEESQNQQENNEKDLLALD
SEQ ID NO: 23- >426c_ZM_RC1_triple.IP (an IP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWC
QSLKPCVKLTPLCV TLQCTNYAPNLLSNMRGELKQCSFNMTTELRDKKRKVNVLFYKL
DLVPLTNSSNTTNYRLISCNTSTITQACPKVTFDPIPIHYCAPAGYAILKCNNKTFNGKGP
CNNVSTVQCTHGIKPVVSTQLLLNGSLAEEEIVIRSKDLSDNAKIIIVQLNTPVQINCTRPN
NNTRKSIRIGPGQSFYATGEIIGDIRMAHCNVSKATWNEAVNQVKKKLKEHFPHKNISFQ
SSSGGDLEITTHSFNCGGEFFYCNTSGLENDTISNATIVLPCRIKQIINMWQEVGCGAMYA
PPIQGVIRCVSNITGLLLLRDGGDTTDNTEIFRPGGGDMRDNWRSELYKYKVVEIKPLGV
APTDAKRRVVERRRRRRAVGIGAVFLGFLGAAGSTMGAASITLTVQARQLLSGIVQQQS
NLLRAPEAQQHMLQLTVWGIKQLQTRVLAIERYLKDQQLLGLWGCSGKLICTTAVPWN
ISWSNKSKEEIWENMTWMQWDREINNYTNTIYRLLEESQNQQENNEKDLLALD
SEQ ID NO: 24- >426c_Q23_RC1_triple.IP (an IP trimer)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWC
QSLKPCVKLTPLCVTLHCTNVTPNLNTTGDREGLKNCSFNMTTELRDKRQKVYSLFYRL
DIVPINENQGSEYRLINCNTSAITQACPKVTFDPIPIHYCAPAGYAILKCNNKTFNGKGPC
NNVSTVQCTHGIKPVVSTQLLLNGSLAEEEIVIRSKDLSDNAKIIIVQLNKSVQINCTRPN
NNTVKSIRIGPGQAFYYFGDIIGDIRMAYCNISGRNWSEAVNQVKKKLKEHFPHKNISFQ
SSSGGDLEITTHSFNCGGEFFYCNTSGLENDTISNDSIMLPCRIKQIINMWQEVGCGAIYA
PPIPGVITCKSDITGLLLLRDGGDTTDNTEIFRPGGGDMRDNWRSELYKYKVVEIKPLGV
APTDAKRRVVERRRRRRAVGIGAVFLGFLGAAGSTMGAASITLTVQARQLLSGIVQQQS
NLLRAPEAQQHMLQLTVWGIKQLQTRVLAIERYLKDQQLLGLWGCSGKLICTTAVPWN
ISWSNKSKEEIWENMTWMQWDREINNYTNTIYRLLEESQNQQENNEKDLLALD
SEQ ID NO: 25 >426c (WT.1-745)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLNCTNVNVTSNSTNVNSSSTDNTTLGEIKNCSFNITTEIRDKTRKE
YALFYRLDIVPLDNSSNPNSSNTYRLINCNTSTLTQACPKVTFDPIPIHYCAPAGYAILKC
NNKTFNGKGPCNNVSTVQCTHGIKPVVSTQLLLNGSLAEEEIVIRSKNLSDNAKIIIVQLN
KSVEIVCTRPNNNTRRSIRIGPGQTFYATDIIGDIRQAYCNISGRNWSEAVNQVKKKLKE
HFPHKNISFQSSSGGDLEITTHSFNCGGEFFYCNTSGLFNDTISNATIMLPCRIKQIINMWQ
EVGKAIYAPPIKGNITCKSDITGLLLLRDGGNTTNNTEIFRPGGGDMRDNWRSELYKYK
VVEIKPLGVAPTDAKRRVVERNKRAVGIGAVFLGFLGAAGSTMGAASITLTVQARQLLS
GIVQQQSNLLRAIEAQQHMLQLTVWGIKQLQTRVLAIERYLKDQQLLGLWGCSGKLICT
TAVPWNISWSNKSKEEIWENMTWMQWDREINNYTNTIYRLLEESQNQQENNEKDLLAL
DSWNNLWNWFNITNWLWYIKIFIMIVGGLIGLRIIFIVLSIVNRVRQGYSPLSFQTLIPNPR
GPDRPGGIEEEGGEQDRDRS
SEQ ID NO: 26 >426c (276D.460.463D.1-745)
MRVRGILRNWQQWWIWGILGFWMLMICSVVGNLWVTVYYGVPVWKEAKTTLFCASD
AKAYEKEVHNVWATHACVPTDPNPQEVVLENVTENFNMWKNDMVDQMQEDVISIWD
QSLKPCVKLTPLCVTLNCTNVNVTSNSTNVNSSSTDNTTLGEIKNCSFNITTEIRDKTRKE
YALFYRLDIVPLDNSSNPNSSNTYRLINCNTSTLTQACPKVTFDPIPIHYCAPAGYAILKC
NNKTFNGKGPCNNVSTVQCTHGIKPVVSTQLLLNGSLAEEEIVIRSKDLSDNAKIIIVQLN
KSVEIVCTRPNNNTRRSIRIGPGQTFYATDIIGDIRQAYCNISGRNWSEAVNQVKKKLKE
HFPHKNISFQSSSGGDLEITTHSFNCGGEFFYCNTSGLENDTISNATIMLPCRIKQIINMWQ
EVGKAIYAPPIKGNITCKSDITGLLLLRDGGDTTDNTEIFRPGGGDMRDNWRSELYKYK
VVEIKPLGVAPTDAKRRVVERNKRAVGIGAVFLGFLGAAGSTMGAASITLTVQARQLLS
GIVQQQSNLLRAIEAQQHMLQLTVWGIKQLQTRVLAIERYLKDQQLLGLWGCSGKLICT
TAVPWNISWSNKSKEEIWENMTWMQWDREINNYTNTIYRLLEESQNQQENNEKDLLAL
DSWNNLWNWFNITNWLWYIKIFIMIVGGLIGLRIIFIVLSIVNRVRQGYSPLSFQTLIPNPR
GPDRPGGIEEEGGEQDRDRS
SEQ ID NO: 27 >WITO.33
MKVMGTKKNYQHLWRWGIMLLGMLMMSSAAEQLWVTVYYGVPVWREANTTLFCAS
DAKAYDTEVHNVWATHACVPTDPNPQEVVMGNVTEDFNMWKNNMVEQMHEDIISLW
DQSLKPCVKLTPLCVTLHCTNVTISSTNGSTANVTMREEMKNCSFNTTTVIRDKIQKEYA
LFYKLDIVPIEGKNTNTGYRLINCNTSVITQACPKVSFEPIPIHYCAPAGFAILKCNNKTFN
GKGPCRNVSTVQCTHGIKPVVSTQLLLNGSLAEEDIIIRSENFTNNGKNIIVQLKEPVKINC
TRPGNNTRRSINIGPGRAFYATGAIIGDIRKAHCNISTEQWNNTLTQIVDKLREQFGNKTII
FNQSSGGDPEVVMHTFNCGGEFFYCNSTQLFNSTWENNGTSTWNSTADNITLPCRIKQV
INMWQEVGKAMYAPPIRGQIDCSSNITGLILTRDGGSNSSQNETFRPGGGNMKDNWRSE
LYKYKVVKIEPLGIAPTRAKRRVVQREKRAVTLGAVFLGFLGAAGSTMGAASLTLTVQ
ARLLLSGIVQQQSNLLRAIEAQQHMLQLTVWGIKQLQARVLAIERYLKDQQLLGIWGCS
GKLICTTTVPWNTSWSNKSYDYIWNNMTWMQWEREIDNYTGFIYTLIEESQNQQEKNE
LELLELDKWASLWNWFNITNWLWYIKLFIMIIGGLVGLRIVCAVLSIVNRVRQGYSPLSF
QTRLPNPRGPDRPEETEGEGGERDRDRSARLVNGFLAIIWDDLRSLCLFSYHRLRDLLLI
VARVVEILGRRGWEILKYWWNLLKYWSQELKNSAVSLLNVTAIAVAEGTDRVIEIVQR
AVRAILHIPTRIRQGFERALL
SEQ ID NO: 28 >Q23.17.gp160
MRVMGIQRNCQHLLTWGIMILGTIIFCSAVENLWVTVYYGVPVWRDADTTLFCASDAK
AYETEKHNVWATHACVPTDPNPQEIHLDNVTEKFNMWKNNMVEQMHTDIISLWDQSL
KPCVKLTPLCVTLHCTNVTSVNTTGDREGLKNCSFNMTTELRDKRQKVYSLFYRLDIVP
INENQGSEYRLINCNTSAITQACPKVSFEPIPIHYCTPAGFAILKCKDEGFNGTGLCKNVST
VQCTHGIKPVVSTQLLLNGSLAEKNITIRSENITNNAKIIIVQLVQPVTIKCIRPNNNTRKSI
RIGPGQAFYATGDIIGDIRQAHCNVTRSRWNKTLQEVAEKLRTYFGNKTIIFANSSGGDL
EITTHSFNCGGEFFYCNTSGLFNSTWYVNSTWNDTDSTQESNDTITLPCRIKQIINMWQR
AGQAMYAPPIPGVIKCESNITGLLLTRDGGKDNNVNETFRPGGGDMRDNWRSELYKYK
VVEIEPLGVAPTRAKRRVVEREKRAVGIGAVFLGFLGAAGSTMGAASITLTVQARQLLS
GIVQQQNNLLRAIEAQQHLLKLTVWGIKQLQARVLAVERYLRDQQLLGIWGCSGKLICT
TNVPWNSSWSNKSLDEIWNNMTWLQWDKEINNYTQLIYRLIEESQNQQEKNEKELLEL
DKWANLWSWFDISNWLWYIKIFIIIVGGLIGLRIVFAVLSVINRVRQGYSPLSFQTHTPNP
RGLDRPERIEEEDGEQGRGRSIRLVSGFLALAWDDLRSLCLFSYHRLRDFILIAARTVELL
GHSSLKGLRLGWEGIKYLWNLLSYWGRELKISAINLVDTIAIAVAGWTDRVIEIAQRIGR
AILHIPVRIRQGLERALL
SEQ ID NO: 29 >ZM233
MRVRGIMRNWQQWWIWGSLGFWMLIICNVMGSLWVTVYYGVPVWREAKTTLFCASD
AKAYETEAHSVWATHACVPTDPNPQEMVLENVTENFNMWKNDMVDQMHEDVISIWD
QSLKPCVKLTPLCVTLDCSTYNNTHNISKEMKICSFNMTTELRDKKRKVNVLFYKLDLV
PLTNSSNTTNYRLISCNTSTITQACPKVSFDPIPIHYCAPAGYAILKCNNKTFNGTGPCNN
VSTVQCTHGIKPVVSTQLLLNGSLAEEEIIIRFENLTDNVKIIIVQLNETINITCTRPNNNTR
KSIRIGPGQSFYATGEIVGNIREAHCNISASKWNKTLERVRTKLKEHFPNKTIEFEPSSGG
DLEITTHSFNCGGEFFYCNTSGLFNSAINGTLTSNVTLPCRIKQIINMWQEVGRAMYAPPI
AGNITCKSNITGLLLTRDGGENSSSTTETFRPTGGDMKNNWRSELYKYKVVEIKPLGIAP
TEAKRRVVEREKRAVGIGAVFLGFLGAAGSTMGAASMTLTVQARQLLSGIVQQQSNLL
KAIEAQQHMLQLTVWGIKQLQARVLAIERYLKDQQLLGLWGCSGKLICTTNVPWNAS
WSNKSKNDIWDNMTWMQWDREISNHTDTIYRLLEDSQNQQEKNEKDLLALDSWKNL
WNWFSITKWLWYIKIFIMIVGGLIGLRIIFAVLSIVNRVRQGYSPLSFQTLTPNPRGPDRLG
GIEEEGGEQDKNKSRRLVTGFLPVVWDDLRSLCLFSYHLLRDFILIVARTVELLGRRGWE
ALKYLGGLVQYWGLELKKSTISLLDTIAIVVAEGTDRIIEVLQRIGRAIYNIPRRIRQGFET
ALL

1. An HIV-1 Env-derived immunogen that simultaneously engages more than one germline lineage of broadly neutralizing antibodies (bNAbs), wherein each of the bNABs exhibits specificity for a distinct neutralization site of the HIV-1 envelope.

2. The HIV-1 Env-derived immunogen of claim 1, wherein a distinct neutralization site comprises the CD4-binding site (CD4-BS), the V2 glycan, the V3 glycan supersite, the gp120/gp41 interface, the fusion peptide region, or the MPER domain of gp41 of the HIV-1 envelope.

3. The HIV-1 Env-derived immunogen of claim 1, wherein a first germline lineage of bNAbs is selected from the group consisting of N6, 3BNC117, CH103, CH235, VRC01, and NIH45-46.

4. The HIV-1 Env-derived immunogen of claim 1, wherein a second germline lineage of bNAbs is selected from the group consisting of CH01, PG9, PG16, PGT1435, and VRC26.

5. The HIV-1 Env-derived immunogen of claim 1, comprising a chimeric polypeptide comprising (a) a first polyamino acid domain representing the “core” domain of a native HIV-1 Env polypeptide selected from the group consisting of CH505 and 426c and (b) a second polyamino acid domain representing the V1-V2-V3 loop regions of a native HIV-1 Env polypeptide selected from the group consisting of CAP256, Q23, WITO, A244, ZM233, and AG_250.

6. The HIV-1 Env-derived immunogen of claim 5, wherein the 426c Env is 426c-deglyco3 (N267D/N460D/N463D).

7. The HIV-1 Env-derived immunogen of claim 5, wherein the Q23 Env is Q23.17.

8. The HIV-1 Env-derived immunogen of claim 1, which is a membrane-bound protein.

9. The HIV-1 Env-derived immunogen of claim 8, which comprises an amino acid sequence represented by one of SEQ ID NOs: 1-8.

10. The HIV-1 Env-derived immunogen of claim 1, which is a soluble protein.

11. The HIV-1 Env-derived immunogen of claim 10, which is a SOSIP trimer.

12. The HIV-1 Env-derived immunogen of claim 10 or 11, which comprises an amino acid sequence represented by one of SEQ ID NOs: 9-16.

13. The HIV-1 Env-derived immunogen of claim 10, which is an IP trimer.

14. The HIV-1 Env-derived immunogen of claim 10, which comprises an amino acid sequence represented by one of SEQ ID NOs: 17-24.

15. A nucleic acid molecule encoding the HIV-1 Env-derived immunogen of claim 5.

16. A genetic vector comprising the nucleic acid molecule of claim 15.

17. A cell comprising the HIV-1 Env-derived immunogen of claim 8.

18. A pharmaceutical composition comprising (a) the HIV-1 Env-derived immunogen of claim 1, (b) a nucleic acid molecule encoding (a), (c) a genetic vector comprising the nucleic acid molecule of, or (d) a cell comprising (a), and a pharmaceutically acceptable carrier.

19. The pharmaceutical composition of claim 18, which comprises an immunostimulatory/regulatory molecule.

20. The pharmaceutical composition of claim 19, wherein the immunostimulatory/regulatory molecule is selected from the group consisting of interleukin (IL)-2, IL-4, IL-6, IL-7, IL-12 (including, optionally, NHS-IL12), IL-15, IL-15/IL-15Ra, IL-15/IL-15Ra-Fc, interferon (IFN)-γ, tumor necrosis factor (TNF)-α, B7.1, B7.2, ICAM-1, ICAM-2, LFA-1, LFA-2, LFA-3, CD70, CD-72, RANTES, G-CSF, GM-CSF, OX-40L, 41 BBL, anti-CTLA-4, IDO inhibitor, anti-PDL1, anti-PD1, and combinations thereof.

21. A method of vaccinating a human patient against HIV comprising administering the pharmaceutical composition of claim 18 to the patient under conditions sufficient for the patient to mount an immune response against HIV.

22. (canceled)

23. The method of claim 21, wherein the HIV is HIV-1.

24. The method of claim 21, wherein the pharmaceutical composition is administered in accordance with a prime/boost vaccination regimen comprising at least one prime vaccination followed by at least one boost vaccination, and wherein the pharmaceutical composition comprises the initial vaccination.

25. The method or of claim 24, comprising administering prime doses at day 0 and 14, wherein the dose at day 14 exceeds that of the dose administered at day 0.

26. The method of claim 24, wherein the vaccination regimen comprises a prime vaccination followed by boost vaccinations at intervals of from 1-10 months.

27. The method of claim 26, wherein the boost vaccinations are at intervals of two months.

28. The method of claim 24, wherein the first boost dose is administered at least four weeks following the prime dose.

29. The method of claim 24, wherein the vaccination regimen comprises an initial prime vaccination employing the pharmaceutical composition, two additional prime vaccinations separated by two-month intervals at two and four months following the initial prime vaccination, and boost vaccinations at six and ten months following the initial prime vaccination.

30. The method of claim 24, comprising administering an immunostimulatory/regulatory molecule to the patient.

31. The method of claim 30, wherein the immunostimulatory/regulatory molecule is selected from the group consisting of interleukin (IL)-2, IL-4, IL-6, IL-7, IL-12 (including, optionally, NHS-IL12), IL-15, IL-15/IL-15Ra, IL-15/IL-15Ra-Fc, interferon (IFN)-γ, tumor necrosis factor (TNF)-α, B7.1, B7.2, ICAM-1, ICAM-2, LFA-1, LFA-2, LFA-3, CD70, CD-72, RANTES, G-CSF, GM-CSF, OX-40L, 41 BBL, anti-CTLA-4, IDO inhibitor, anti-PDL1, anti-PD1, and combinations thereof.
